(19) United States
(12) Patent Application Publication
(10) Pub. No.: US 2012/0079613 A1
(43) Pub. Date: Mar. 29, 2012

(54) Homeobox Transcription Factor BSX and Uses Thereof for Treating Diseases, in Particular Obesity

(75) Inventors: Mathias Treier, Schwetzingen (DE); Maria Sakkou, Munich (DE); Petra Wiedmer, Potsdam (DE); Matthias Tschoep, Cincinnati, OH (US)

(73) Assignees: Deutsches Institut fur Ernahrungsforschung (DIFE), Nuthetal (DE); European Molecular Biology Laboratory (EMBL), Heidelberg (DE)

(21) Appl. No.: 12/296,814

(22) PCT Filed: Apr. 2, 2007
(86) PCT No.: PCT/EP07/02957

§ 371 Date: Sep. 16, 2009

Related U.S. Application Data
(60) Provisional application No. 60/790,660, filed on Apr. 10, 2006.

(51) Int. Cl. A61K 39/395; C12N 15/12; C07K 16/18; A61K 38/17; A61K 31/711; C07K 1/14; A61P 3/04; C12N 15/85; A01K 67/027; C12N 5/10; C12P 21/02; C12P 19/34; C07K 14/00; A61K 31/7105

(52) U.S. Cl. 800/13; 530/350; 536/23.5; 530/387.9; 514/4.8; 514/44 R; 424/139.1; 435/320.1; 435/325; 435/69.1; 435/91.1

(57) ABSTRACT

The present invention relates to the human homeobox transcription factor Bsx protein, nucleic acids encoding for said protein, vectors and cells comprising the nucleic acids, antibodies directed against Bsx, and methods for identifying compounds binding to Bsx, as well as to the use of proteins, nucleic acids, vectors, cells, and interacting compounds for treating obesity and related diseases.

The present invention relates to the human homeobox transcription factor Bsx protein, nucleic acids encoding for said protein, vectors and cells comprising the nucleic acids, antibodies directed against Bsx, and methods for identifying compounds binding to Bsx, as well as to the use of proteins, nucleic acids, vectors, cells, and interacting compounds for treating obesity and related diseases.

The arcuate nucleus (ARC) of the hypothalamus harbours two distinct neuronal populations, on which central and peripheral signals of energy stores converge. The orexigenic neuropeptide Y/agouti-related peptide (NPY/AgRP) neurons promote weight gain and are directly inhibited by leptin, whereas the anorexigenic pro-opiomelanocortin (POMC) neurons inhibit food intake and promote weight loss in a leptin-dependent manner by releasing a-melanocyte stimulating hormone (aMSH), a product of POMC processing.

Additional satiety and hunger signals sensed by these neurons include ghrelin, glucose, insulin and peptide YY. Both neuronal populations send dense projections to second order neurons involved in energy metabolism located in the paraventricular nucleus (PVN), zona incerta, perifornical area and lateral hypothalamic area. NPY/AgRP neurons directly inhibit POMC neurons and antagonize the action of aMSH on melanocortin-4 receptors (MC4R) via the release of AgRP.

Mutations that abolish the production of leptin, leptin receptor, MC4R or POMC lead to obesity in mice and man. In contrast, mutations that prevent the expression of NPY and/or AgRP have close to no impact on feeding behavior, despite the fact that application of NPY or AgRP intracranially leads to a robust feeding response and increased energy expenditure in rodents, respectively.

These observations have raised the question if NPY/AgRP neurons are indeed important for the regulation of body weight although loss of NPY leads to the attenuation of the obesity syndrome of leptin deficient ob/ob mice. Recent studies have now firmly established that NPY/AgRP neurons are essential for feeding in adult mice.

One in three or 58 million American adults aged 20 through 74 are overweight. According to data from the Third National Health and Nutrition Examination Survey (NHANES III), the number of overweight Americans increased from 25 to 33 percent between 1980 and 1991. Even using a more rigorous definition recommended for youths, 11 percent of children and adolescents are overweight, up from approximately 5 percent in the 1960s and 70s. Overweight and obesity is a known risk factor for diabetes, heart disease, high blood pressure, gallbladder disease, arthritis, breathing problems, and some forms of cancer.

Recent discoveries have helped explain how genes may determine obesity and how they may influence the regulation of body weight. For example, as discussed above, mutations in the ob gene have led to massive obesity in mice. Cloning the ob gene led to the identification of leptin, a protein coded by this gene; leptin is produced in adipose tissue cells and acts to control body fat. The existence of leptin supports the idea that body weight is regulated, because leptin serves as a signal between adipose tissue and the areas of the brain that control energy metabolism, which influences body weight.

The extent of genetic influences on human obesity has been assessed by twin, adoption, and family studies. In the first studies, of twins, the heritability of the body-mass index (BMI) was estimated to be very high, about 80%, and this value is still frequently cited. The results of adoption and family studies, however, agree on a heritability of about 33%, which is generally viewed as more reasonable than that of the twin studies. Genetic influences may be more important in determining regional fat distribution than total body fat, particularly the critical visceral fat depot.

Current treatments of obesity include surgery and drug therapy. Nevertheless, in particular due to complications with side effects of some medications, such as fenfluramine alone or in combination with phentermine, novel drugs and treatments are required.

Bsx is an evolutionary conserved Brain Specific homeoboX gene expressed in the septum, epiphysis, mammillary bodies and arcuate nucleus. During a search for novel homeobox genes in the public released genomic sequences derived by the Human and Mouse genome projects, Cremona et al. were able to identify the mouse homologue of the Drosophila brain specific homeobox gene, but failed to provide the human sequence (Cremona M, Colombo E, Andreazzoli M, Cossu G, Broccoli V. Bsx, an evolutionary conserved Brain Specific homeoboX gene expressed in the septum, epiphysis, mammillary bodies and arcuate nucleus. Gene Expr Patterns. 2004 January; 4(1):47-51). Furthermore, the expression of Bsx in embryonic and post-natal mouse brain was characterized. Interestingly, Bsx showed an expression pattern restricted to a few specific developing brain structures. Pineal gland, telencephalic septum, hypothalamic pre-mammillary body and arcuate nucleus are the only brain structures where the inventors detected Bsx transcriptional activity, which is maintained also after birth. Cremona et al. concluded that Bsx might be considered an important molecular marker for early embryonic stages of epiphysis development, being specifically expressed in this neural structure from E9.5 onwards.

Obesity genetic markers and their uses in screening assays and respective diagnostic and treatment approaches (e.g. siRNA) are well known in the patent literature, for example from WO 2005-123949, WO 2005-123948, WO 2005-111239, WO 2005-021787, WO 2004-092411, WO 03-097683, WO 03-070968, WO 03-070885, WO 03-070881, WO 03-016553, WO 02-055694, WO 02-42324, WO 02-29097, WO 02-33063, WO 01-94605, WO 01-82921, WO 00-09686, and WO 99-67407.

Body weight remains stable when food intake and energy expenditure are in caloric balance. Energy expenditure can be partitioned into three major categories: basic cellular and physiological functions that require ATP (resting thermogenesis), the thermic effect of food, and thermogenesis induced by physical activity (Castaneda, T. R., Jurgens, H., Wiedmer, P., Pfluger, P., Diano, S., Horvath, T. L., Tang-Christensen, M., and Tschop, M. H. (2005). Obesity and the neuroendocrine control of energy homeostasis: the role of spontaneous locomotor activity. J Nutr 135, 1314-1319; Tou, J. C., and Wade, C. E. (2002). Determinants affecting physical activity levels in animal models. Exp Biol Med (Maywood) 227, 587-600). Although physical activity expends calories, increased physical activity is essential for food acquisition. Thus, molecular mechanisms should exist that integrate these complex behaviours in the service of energy homeostasis. Consistent with that, both MCH and hypocretin/orexin are important in the control of locomotory behavior (Georgescu, D., Sears, R. M., Hommel, J. D., Barrot, M., Bolanos, C. A., Marsh, D. J., Bednarek, M. A., Bibb, J. A., Maratos-Flier, E., Nestler, E. J., and DiLeone, R. J. (2005). The hypothalamic neuropeptide melanin-concentrating hormone acts in the nucleus accumbens to modulate feeding behavior and forced-swim performance. J Neurosci 25, 2933-2940; Marsh, D. J., Weingarth, D. T., Novi, D. E., Chen, H. Y., Trumbauer, M. E., Chen, A. S., Guan, X. M., Jiang, M. M., Feng, Y., Camacho, R. E., et al. (2002). Melaninconcentrating hormone 1 receptor-deficient mice are lean, hyperactive, and hyperphagic and have altered metabolism. Proc Natl Acad Sci USA 99, 3240-3245; Thorpe, A. J., and Kotz, C. M. (2005). Orexin A in the nucleus accumbens stimulates feeding and locomotor activity. Brain Res 1050, 156-162), and leptin signaling in the arcuate is necessary to maintain normal physical activity (Coppari, R., Ichinose, M., Lee, C. E., Pullen, A. E., Kenny, C. D., McGovern, R. A., Tang, V., Liu, S. M., Ludwig, T., Chua, S. C., Jr., et al. (2005). The hypothalamic arcuate nucleus: a key site for mediating leptin's effects on glucose homeostasis and locomotor activity. Cell Metab 1, 63-72).

It is therefore an object of the present invention, to provide an additional new option for the diagnosis and treatment of obesity, together with respective medical and diagnostic tools and uses thereof.

Therefore, the present invention is directed at an isolated protein comprising the same or substantially the same amino acid sequence of human Bsx homeobox transcription factor (depicted in SEQ ID NO: 1), or a splice variant or a salt thereof. A protein having substantially the same amino acid sequence comprises proteins with at least about 95%, preferably at least about 96%, more preferably at least about 97%, more preferably with at least about 98% and most preferably with at least about 99% amino acid sequence identity. The amino acid exchanges are preferably so called conservative changes meaning substitutions of, for example, a polar amino acid residue by another polar amino acid residue, of an acidic amino acid residue by another acidic amino acid residue or of a basic amino acid residue by another basic amino acid residue.

Within the context of the present invention it has been found that neuropeptide Y (NPY) and agouti-related protein (AgRP) expressing neurons in the arcuate nucleus of the hypothalamus are essential for feeding behaviour and regulation of body weight. The inventors could show that the evolutionarily conserved brain-specific homeobox transcription factor Bsx, expressed in NPY/AgRP neurons, is required for Npy and Agrp expression and voluntary locomotion. In addition, NPY/AgRP neurons lacking Bsx function are defective in their response to starvation and ghrelin signalling. Furthermore, the inventors demonstrate that loss of Bsx attenuates the obesity syndrome in ob/ob mice caused by leptin-deficiency. Surprisingly, mice mutant for both, Bsx and leptin, do not show improved locomotor activity providing evidence that the proposed inverse correlation of body weight and physical activity can be genetically uncoupled. These results suggest that physical activity is a contributor rather than a response to weight gain.

The orphan homeobox in Bsx is highly conserved in Drosophila, C. elegans and mammals suggesting that Bsx is at the basis of an evolutionary conserved system required for food acquisition and body weight regulation. Although counter productive with respect to energy saving, it makes perfect sense that hunger, signalled i.e. by ghrelin is coupled to increased locomotor activity as animals have to move in their drive to find food. However, once food is secured short-term satiety signals like PYY, PP and possible NPY itself may decrease physical activity to save energy. Whereas mutations leading to decreased locomotor activity are disadvantages for the survival of animals, our society structure no longer counterselects in these cases.

The possibility to study the regulation of Bsx activity and the connected transcriptional network underlying NPY/AgRP neuron physiology may allow to stimulate locomotor activity which would open new avenues in the fight against obesity.

Proteins having substantially the same amino acid sequence within the meaning of this invention exhibit in a preferred embodiment homeobox transcription factor activity. The homeobox transcription factor activity of a given protein with substantially the same amino acid sequence can be tested, for example, by a suitable assay described in the examples below. The proteins employed in the assay can either be purified from cells or can be recombinantly expressed and purified by methods well known in the art.

In one embodiment of the present invention the protein comprises at least one fragment of the human Bsx. A fragment within the meaning of the present invention refers to one of the proteins according to SEQ ID NO: 1 bearing at least one N-terminal, C-terminal and/or internal deletion. The resulting fragment has a length of at least about 50, preferably of at least about 100, more preferably of at least about 150, more preferably of at least about 200, more preferably of at least about 250, more preferably of at least about 300 and in case of human Bsx, more preferably of at least about 350 and most preferably of at least about 400 amino acids.

In a further aspect the present invention is directed at a nucleic acid, which comprises at least one nucleic acid encoding one of the proteins of the present invention or the regulatory regions of Bsx according to SEQ ID Nos. 68 to 76 or the homologs thereof of other mammalian species. Preferably, the nucleic acid consists of DNA, CNA, PNA, or RNA, wherein the DNA preferentially is either single or double stranded. Also comprised are DNA's, which hybridize to one of the aforementioned DNA's preferably under stringent conditions like, for example, hybridization at 60° C. in 2.5×SSC buffer and several washes at 37° C. at a lower buffer concentration like, for example, 0.5×SSC buffer and which encode proteins exhibiting homeobox transcription factor activity. Additional reagents required for carrying out stringent Northern or Southern blots like, for example, single stranded salmon sperm DNA are well known in the art. Also comprised are nucleic acid sequences, which are related to the nucleic acid according to SEQ ID No. 2 and/or the hybridizing nucleic acids as outlined above by the degeneration of the genetic code.

In a preferred embodiment of the nucleic acid of the present invention the nucleic acid comprises a nucleic acid selected from the group consisting of the human Bsx gene (see SEQ ID NO: 2). Another preferred embodiment is directed to a nucleic acid of the present invention that comprises a nucleic acid of SEQ ID NO: 2, in that it essentially consists of a nucleic acid of SEQ ID NO: 2.

In a further embodiment the nucleic acid of the present invention further comprises at least one promoter, enhancer, intron and/or polyA-sequence. Preferred promoters or enhancers have tissue specificity, such as neuronal specificity.

In a further embodiment the nucleic acid of the present invention further comprises the regulatory regions of the gene Bsx comprising a sequence according to SEQ ID Nos. 68 to 76 or the homologs thereof of other mammalian species. These regions have been identified as extremely conserved between mammalian species (in particular human, mouse and rat), and are used to interfere with the regulation of the expression of Bsx and/or are used for diagnostic approaches (see below). As an example, regulatory region 3 contains a conserved stat binding site, indicating a leptin-mediated regulation of Bsx. The regulatory regions can be used in order to design oligonucleotides as detailed below, but can also be used in order to screen proteins that bind to motifs inside said sequences. Methods for such assays are well known to the person of skill.

In some instances it might be desirable to interfere with, for example, the transcription or translation of the nucleic acids of the present invention and, therefore, the present invention is also directed at a nucleic acid, which is complementary to the nucleic acid of the present invention and, thus, is capable of inhibiting, for example, transcription or translation. A preferred embodiment of such a complementary nucleic acid is a so called anti-sense oligonucleotide (R. Q. Zheng and D. M. Kemeny (1995) Clin. Exp. Immunol. 100:380-2, W. Nellen and C. Lichtenstein (1993) Trends. Biochem. Sci. 18:419-423 and C. A. Stein (1992) Leukemia 6:967-74), ribozymes (M. Amarzguioui and H. Prydz (1998) Cell. Mol. Life. Sci. 54:1175-1202, N. K. Vaish et al (1998) Nucleic Acids Res. 96:5237-5242, Persidis (1997) Nat. Biotechnol. 15:921-922 and L. A. Couture and D. T. Stinchcomb (1996) Trends Genet. 12:510-515) and/or so called small interfering RNA-molecules (siRNAs or RNAi's) (S. M. Elbashir et al. (2001) Nature 411:494-498, Rondinone CM. RNAi for the Identification of New Targets for the Treatment of Metabolic Diseases. Endocrinology. 2006 March 23). Anti-sense oligonucleotides are able to decrease the stability of the above described nucleic acids and/or can inhibit the translation. Similarly the use of siRNA-oligonucleotides can also lead to a reduction in the amount of the translated polypeptides. Anti-sense oligonucleotides have in a preferred embodiment a length of at least 20, preferable of at least about 30, more preferably of at least about 40 and most preferably a length of at least about 50 nucleic acids.

Oligonucleotides are generally rapidly degraded by endo- or exonucleases, which are present in the cell, in particular by DNases and RNases and, therefore, it is advantageous to modify the nucleic acids which are used, for example, in anti-sense strategies, as ribozymes or siRNAs to stabilize them against degradation and thereby prolong the time over which an effective amount of the nucleic acid is maintained within the cell (L. Beigelmann et al. (1995) Nucleic Acids Res. 23:3989-94, WO 95/11910, WO 98/37340 and WO 97/29116). Typically such stabilization can be obtained by the introduction of one or more internucleotide phosphate groups and/or by the introduction of one or more non-phosphor-internucleotides.

Suitable modified internucleotides are summarized in, for example, Uhlmann and Peimann (1990) Can. Rev. 90:544. Modified internucleotide phosphate residues and/or non-phosphate bridges which can be used in a nucleic acid of the invention comprise, for example, methylphosphonate, phosphorthioate, phosphoramidate, phosphordithionate, phosphate ester, non-phosphor internucleotide analogues, which can be used in nucleic acids of the invention include, for example, siloxane bridges, carbonate bridges, carboxymethylester, acetamide bridges and/or thioether bridges.

A further aspect of the present invention is directed at a vector comprising a protein according to the present invention and/or a nucleic acid according to the present invention. A vector within the meaning of the present invention is a protein or a nucleic acid or a mixture thereof which is capable of being introduced or of introducing the proteins and/or nucleic acid comprised into a cell. It is preferred that the proteins encoded by the introduced nucleic acid are expressed within the cell upon introduction of the vector.

In a preferred embodiment the vector of the present invention comprises plasmids, phagemids, phages, cosmids, artificial mammalian chromosomes, knock-out or knock-in constructs, viruses, in particular adenovirus, vaccinia virus, lentivirus (Chang, L. J. and Gay, E. E. (20001) Curr. Gene Therap. 1:237-251), Herpes simplex virus (HSV-1, Carlezon, W. A. et al. (2000) Crit. Rev. Neurobiol.), baculovirus, retrovirus, adeno-associated-virus (AAV, Carter, P. J. and Samulski, R. J. (2000) J. Mol. Med. 6:17-27), rhinovirus, human immune deficiency virus (HIV), filovirus and engineered versions thereof (see, for example, Cobinger G. P. et al (2001) Nat. Biotechnol. 19:225-30), virosomes, “naked” DNA liposomes, and nucleic acid coated particles, in particular gold spheres. Particularly preferred are viral vectors like adenoviral vectors or retroviral vectors (Lindemann et al. (1997) Mol. Med. 3:466-76 and Springer et al. (1998) Mol. Cell. 2:549-58). Liposomes are usually small unilamellar or multilamellar vesicles made of neutral cationic and/or anionic lipids, for example, by ultrasound treatment of liposomal suspensions. The DNA can, for example, be ionically bound to the surface of the liposomes or internally enclosed in the liposome. Suitable lipid mixtures are known in the art and comprise, for example, cholesterol, phospholipide like, for example, phosphatidylcholin (PC), phosphatidylserin (PS) and the like, DOTMA (1,2-Dioleyloxpropyl-3-trimethylammoniumbromid) and DPOE (Dioleoylphosphatidylethanolamin) which both have been used on a variety of cell lines.

Nucleic acid coated particles are another means for the introduction of nucleic acids into cells using so called “gene guns”, which allow the mechanical introduction of particles into the cells. Preferably the particles itself are inert, and therefore, are in a preferred embodiment made out of gold spheres.

In a further aspect the present invention is directed at an isolated host cell comprising a protein of the present invention, a nucleic acid of the present invention and/or a vector of the present invention. Cells of the present invention can be prokaryotic or eukaryotic cells and in a preferred embodiment the cells of the present invention are stem cells, in particular non-human embryonic stem cells, embryonic stem cell lines, foetal stem cells, adult stem cells, neuronal precursor cells or neuronal cells in particular axons (Hsich, G. et al. (2002) Hum. Gene Therap., 13:579-604 and Martinez-Serrano, A. et al. (2001) Curr. Gene Therap. 1:279-299). The cells preferably comprise the nucleic acids extrachromosomally or interchromosomally. Thus, human embryonal germ line cells and human embryos can be excluded.

A further aspect of the present invention is a transgenic non-human animal generated from a cell or cells of the present invention. The animal can be a mosaic animal, which means that only part of the cells making up the body comprise cells of the present invention or the animal can be a transgenic animal which means that all cells of the animal are derived from a cell of the present invention. Mosaic or transgenic animals can be either homo- or heterozygous with respect to the nucleic acid of the present invention contained within the cell of the present invention. In a preferred embodiment the transgenic animals are either homo- or heterozygous knock-out or knock-in animals with respect to the genes which code for the proteins of the present invention.

In a further aspect the present invention is directed at an antibody directed against a protein of the present invention. The term “antibody” comprises monoclonal and polyclonal antibodies and binding fragments thereof, in particular Fc-fragments as well as so called “single-chain-antibodies”(Bird R. E. et al (1988) Science 242:423-6) and diabodies (Holliger P. et al (1993) Proc. Natl. Acad. Sci. U.S.A. 90:6444-8).

In a further aspect the present invention is directed at a method of producing a protein of the present invention or a nucleic acid of the present invention and comprises the steps of: a) cultivating a cell of the present invention and b) isolating the protein and/or the nucleic acid. If the method is used primarily to isolate nucleic acids then in a preferred embodiment the cells, which are used are prokaryotic cells, in particular E. coli cells. If the method is used primarily for the isolation of proteins of the invention than the cells can be either of prokaryotic or eukaryotic origin. Someone of skill in the art is aware of a variety of different cell types suitable for the production of proteins like, for example, E. coli, Sf9, Hi5, P. pastoris, COS and HeLa. Eukaryotic cells are preferably chosen, if it is desired that the proteins produced by the cells exhibit an essentially natural pattern of glycosylation and prokaryotic cells are chosen, if, for example, glycosylation or other modifications, which are normally introduced into proteins only in eukaryotic cells, are not desired or not needed.

In a further aspect the present invention is directed at a method of isolating compounds interacting with a protein of the present invention comprising the steps of: a) contacting one or more of the proteins of the present invention, preferably one, with at least one potentially interacting compound, and b) measuring binding of said compound to said protein. This method is suitable for the determination of compounds that can interact with the proteins of the present invention and to identify, for example, inhibitors, activators, competitors or modulators of proteins of the present invention, in particular inhibitors, activators, competitors or modulators of the enzymatic activity of the proteins of the present invention.

The potentially binding substance, whose binding to the protein of the present invention is to be measured, can be any chemical substance or any mixture thereof. For example, it can be a substance of a peptide library, a combinatory library, a cell extract, in particular a plant cell extract, a “small molecular drug”, a protein and/or a protein fragment.

The term “contacting” in the present invention means any interaction between the potentially binding substance(s) with the proteins of the invention, whereby any of the two components can be independently of each other in a liquid phase, for example in solution, or in suspension or can be bound to a solid phase, for example, in the form of an essentially planar surface or in the form of particles, pearls or the like. In a preferred embodiment a multitude of different potentially binding substances are immobilized on a solid surface like, for example, on a compound library chip and the protein of the present invention is subsequently contacted with such a chip.

The proteins of the present invention employed in a method of the present invention can be full length proteins or a fragments with N/C-terminal and/or internal deletions. Preferably the fragments are either N-terminal fragments comprising the enzymatic region of the protein or C-terminal fragments comprising the cytoplasmic region, depending on whether potentially interacting compounds are sought that specifically interact with the N- or C-terminal fragment.

Measuring of binding of the compound to the protein can be carried out either by measuring a marker that can be attached either to the protein or to the potentially interacting compound. Suitable markers are known to someone of skill in the art and comprise, for example, fluorescence or radioactive markers. The binding of the two components can, however, also be measured by the change of an electrochemical parameter of the binding compound or of the protein, e.g. a change of the redox properties of either the protein or the binding compound, upon binding. Suitable methods of detecting such changes comprise, for example, potentiometric methods. Further methods for detecting and/or measuring the binding of the two components to each other are known in the art and can without limitation also be used to measure the binding of the potential interacting compound to the protein or protein fragments of the present invention. The effect of the binding of the compound or the activity of the protein can also be measured indirectly, for example, by assaying the phosphatase activity of the protein after binding.

As a further step after measuring the binding of a potentially interacting compound and after having measured at least two different potentially interacting compounds at least one compound can be selected, for example, on grounds of the measured binding activity or on grounds of the detected increase or decrease of protein activity, in particular homeobox transcription factor activity upon binding. The homeobox transcription factor activity can be measured, for example, as described below.

The thus selected binding compound is then in a preferred embodiment modified in a further step. Modification can be effected by a variety of methods known in the art, which include without limitation the introduction of novel side chains or the exchange of functional groups like, for example, introduction of halogens, in particular F, Cl or Br, the introduction of lower alkyl groups, preferably having one to five carbon atoms like, for example, methyl, ethyl, n-propyl, isopropyl, n-butyl, isobutyl, tert-butyl, n-pentyl or iso-pentyl groups, lower alkenyl groups, preferably having two to five carbon atoms, lower alkynyl groups, preferably having two to five carbon atoms or through the introduction of, for example, a group selected from the group consisting of NH₂, NO₂, OH, SH, NH, CN, aryl, heteroaryl, COH or COOH group.

The thus modified binding substances are than individually tested with the method of the present invention, i.e. they are contacted with the protein and subsequently binding of the modified compounds to the protein is measured. In this step, both the binding per se can be measured and/or the effect of the function of the protein like, e.g. the enzymatic activity of the protein can be measured. If needed the steps of selecting the binding compound, modifying the binding compound, contacting the binding compound with a protein of the invention and measuring the binding of the modified compounds to the protein can be repeated a third or any given number of times as required. The above described method is also termed “directed evolution” since it involves a multitude of steps including modification and selection, whereby binding compounds are selected in an “evolutionary” process optimizing its capabilities with respect to a particular property, e.g. its binding activity, its ability to activate, inhibit or modulate the activity, in particular the homeobox transcription factor activity of the proteins of the present invention.

In a further embodiment of the method of the present invention the interacting compound identified as outlined above, which may or may not have gone through additional rounds of modification and selection, is admixed with suitable auxiliary substances and/or additives. Such substances comprise pharmacological acceptable substances, which increase the stability, solubility, biocompatibility, or biological half-life of the interacting compound or comprise substances or materials, which have to be included for certain routs of application like, for example, intravenous solution, sprays, Band-Aids or pills.

Since lack expression of Bsx has a positive influence on body weight, these proteins have another utility in the treatment of obesity and related diseases, such as diabetes. Accordingly a further aspect of the present invention is a pharmaceutical composition for the treatment of obesity and related diseases, such as diabetes comprising a protein of the invention, a nucleic acid of the invention, a vector of the invention, a cell of the invention, an antibody of the invention, a binding component isolated by a method of the invention and if needed suitable auxiliary substances and/or additives.

Another aspect of the present invention then relates to a protein according to the invention according to SEQ ID No 1, 3 or 5, a nucleic acid according to the invention according to SEQ ID No 2, 4, 6 or 7 or 68 to 76, a vector containing these nucleic acids as above, a cell according to the invention, an antibody according to the invention, a binding compound isolated by the method according to the invention and/or a pharmaceutical composition according to the invention for use in medicine. All these compounds have not been described as useful in the context of a medical treatment, yet. It shall be understood that the embodiments as described above for SEQ ID Nos 1 and 2 apply mutatis mutandis also for the SEQ ID Nos 3 to 7, and these are included in the scope of the present invention. As a non-limiting example, also a fragment of the polypeptide according to SEQ ID No 3 can be used in medicine. The following table summarizes the SEQ ID Nos of the present invention, together with their origins.

| SEQ ID No | Sequence | Origin |
|---|---|---|
| 1 | Bsx Polypeptide | Human |
| 2 | Bsx Polynucleotide (cDNA) | Human |
| 3 | Bsx Polypeptide | Mouse |
| 4 | Bsx Polynucleotide (cDNA) | Mouse |
| 5 | Bsx Polypeptide | Rat |
| 6 | Bsx Polynucleotide (cDNA) | Rat |
| 7 | Bsx Polynucleotide | Human |
| | (intron/exon-structure) | |
| 68 | Bsx regulatory region 1 | Human |
| 69 | Bsx regulatory region 1 | Mouse |
| 70 | Bsx regulatory region 1 | Rat |
| 71 | Bsx regulatory region 2 | Human |
| 72 | Bsx regulatory region 2 | Mouse |
| 73 | Bsx regulatory region 2 | Rat |
| 74 | Bsx regulatory region 3 | Human |
| 75 | Bsx regulatory region 3 | Mouse |
| 76 | Bsx regulatory region 3 | Rat |

Accordingly, a further aspect of the present invention is the use of a pharmaceutical composition of the invention for the production of a medicament for the treatment of obesity and/or a related disease, such as diabetes. Other diseases which can be treated with the pharmaceutical composition comprise locomotive diseases.

A further aspect of the present invention is then a method of treatment of obesity and/or a related disease or condition, such as diabetes or a locomotive disease or condition, in a mammal, comprising administering to said mammal an effective amount of a protein according to the invention as above, a nucleic acid according to the invention as above, a vector containing these nucleic acids as above, a cell according to the invention as above, an antibody according to the invention as above, a binding compound isolated by the method according to the invention as above and/or a pharmaceutical composition according to the invention as above. Preferably, an inhibiting active agent of Bsx is administered in form of a pharmaceutical composition, such as an antibody, antisense nucleotide or an inhibiting binding compound. Preferably, said mammal is a human being. Treating is meant to include, preventing, reducing the symptoms of, or curing the disease or condition.

An “effective amount” is an amount of the compound(s) as mentioned above that a) acts on the expression and/or abundance of Bsx as analysed, and which alleviates symptoms as found for obesity and/or a related disease or condition, such as diabetes or a locomotive disease or condition. Alleviating is meant to include, preventing, treating, reducing the symptoms of, or curing the disease or condition.

Another aspect of the present invention is the use of the proteins or nucleic acids or the antibodies of the present invention as diagnostic marker for the diagnosis of a disease or disease state, such as obesity and diseases associated therewith, such as diabetes, whereby the presence, the absence, or the amount of Bsx proteins is evaluated by, for example, immunological methods, RT-PCR, Northern blot. For the immunological detection and/or quantification methods, the antibodies of the present invention can be used. One particular aspect of this diagnosis is the analysis of the nucleic acids of the present invention, in particular the regulatory regions of the gene Bsx comprising a sequence according to SEQ ID Nos. 68 to 76 or the homologs thereof of other mammalian species, as diagnostic markers for the diagnosis of a disease or disease state, such as obesity and diseases associated therewith, such as diabetes. Mutations, such as nucleotide polymorphisms in these regions or deletions and/or modified methylation that are identified between diseased (obese) and healthy patients.

That physical activity is beneficial for body weight regulation is undisputed. However if the decrease in locomotor activity observed in overweight people is cause or consequence has remained an open issue. It has been proposed that decreased locomotor activity in genetically obese ob/ob and db/db mice only occurs after they develop their characteristic obesity suggesting the existence of an inverse weight-activity relationship which leads to the idea that white adipose tissue releases a factor that slows physical activity. However, the finding that leptin injection stimulates locomotor activity in ob/ob mice before any benefits for weight reduction can be observed suggests a neuronal circuitry that can instantly influence locomotor activity. The Bsx^{-/-}, ob/ob phenotype resembles that displayed by ob/ob mice with Npy deficiency (ob/ob, Npy−/−) i.e. rescue of hyperglycemia, hyperinsulinemia and fertility of the leptin mutant phenotype resulting in a significant weight reduction with one important difference. Whereas Npy deficiency in the ob/ob background partially restores physical activity, this is not the case in Bsx^{-/-}, ob/ob mice.

Hypothalamic neurons producing NPY and AgRP have recently been confirmed as essential centres of food intake and body weight regulation (Gropp, E., Shanabrough, M., Borok, E., Xu, A. W., Janoschek, R., Buch, T., Plum, L., Balthasar, N., Hampel, B., Waisman, A., et al. (2005). Agouti-related peptideexpressing neurons are mandatory for feeding. Nat Neurosci 8, 1289-1291; Luquet, S., Perez, F. A., Hnasko, T. S., and Palmiter, R. D. (2005). NPY/AgRP neurons are essential for feeding in adult mice but can be ablated in neonates. Science 310, 683-685). The most powerful signals known to activate these arcuate neurons are food deprivation or administration of ghrelin (Cowley, M. A., Smith, R. G., Diano, S., Tschop, M., Pronchuk, N., Grove, K. L., Strasburger, C. J., Bidlingmaier, M., Esterman, M., Heiman, M. L., et al. (2003). The distribution and mechanism of action of ghrelin in the CNS demonstrates a novel hypothalamic circuit regulating energy homeostasis. Neuron 37, 649-661; Hahn, T. M., Breininger, J. F., Baskin, D. G., and Schwartz, M. W. (1998). Coexpression of Agrp and NPY in fasting-activated hypothalamic neurons. Nat Neurosci 1, 271-272). The present data indicate that the homeobox transcription factor Bsx is required for adaptive increases in Npy and Agrp expression, for the activation of NPY/AgRP neurons via the ghrelin receptor, and for a normal hyperphagic response to fasting. Since deficiency for Bsx also rescues the hyperphagia of leptin-deficient mice similar to what has been observed in ob/ob mice with Npy deficiency (Erickson, J. C., Hollopeter, G., and Palmiter, R. D. (1996b). Attenuation of the obesity syndrome of ob/ob mice by the loss of neuropeptide Y. Science 274, 1704-1707), the inventors conclude that Bsx may also be required for non-physiological types of hyperphagia.

Leptin-deficient mice also have decreased locomotor activity, and rescue of leptin signaling in mice deficient for leptin or the leptin receptor normalizes their locomotor activity before any benefits in terms of weight reduction can be observed, pointing toward a body weight-independent neuroendocrine control of spontaneous physical activity (Coppari et al., 2005; Pelleymounter et al., 1995). The existence of a, as of yet unknown, hypothalamic molecular control of locomotor activity would be consistent with the observation that caloric restriction leads to an immediate change in locomotory behaviour of rodents. One endocrine factor that may be involved in these processes is ghrelin, which increases during food deprivation and which modulates locomotor activity (Castaneda, T. R., Jurgens, H., Wiedmer, P., Pfluger, P., Diano, S., Horvath, T. L., Tang-Christensen, M., and Tschop, M. H. (2005). Obesity and the neuroendocrine control of energy homeostasis: the role of spontaneous locomotor activity. J Nutr 135, 1314-1319; Finger, F. W. (1951). The effect of food deprivation and subsequent satiation upon general activity in the rat. J Comp Physiol Psychol 44, 557-564; Novak, C. M., Jiang, X., Wang, C., Teske, J. A., Kotz, C. M., and Levine, J. A. (2005). Caloric restriction and physical activity in zebrafish (Danio rerio). Neurosci Lett 383, 99-104; Wald, G., and Jackson, B. (1944). Activity and Nutritional Deprivation. Proc Natl Acad Sci USA 30, 255-263).

The phenotypic analysis of Bsx-mutant mice indicates that Npy and Agrp expression as well as hypothalamic control of locomotory behaviour depends on Bsx function. Mice deficient for Bsx lose 50% of their spontaneous physical activity and fail to increase home cage activity upon food deprivation. An essential requirement for Bsx function in locomotory behaviour control is further supported by the Bsx^{ΔHD/ΔHD}, ob/ob phenotype, where locomotor activity of ob/ob mice was not increased despite a significant reduction in body weight. That locomotory behaviour and body weight control might rely on overlapping hypothalamic neuronal networks is supported by analysis of other mouse mutants. For example, it is well established that loss of MCH improves locomotor activity in ob/ob mice, and that this reduces their obesity syndrome (Segal-Lieberman, G., Bradley, R. L., Kokkotou, E., Carlson, M., Trombly, D. J., Wang, X., Bates, S., Myers, M. G., Jr., Flier, J. S., and Maratos-Flier, E. (2003a). Melanin-concentrating hormone is a critical mediator of the leptin-deficient phenotype. Proc Natl Acad Sci USA 100, 10085-10090). In addition, disruption of the MCH receptor 1 has been reported to lead to hyperactivity (Marsh, D. J., Weingarth, D. T., Novi, D. E., Chen, H. Y., Trumbauer, M. E., Chen, A. S., Guan, X. M., Jiang, M. M., Feng, Y., Camacho, R. E., et al. (2002). Melaninconcentrating hormone 1 receptor-deficient mice are lean, hyperactive, and hyperphagic and have altered metabolism. Proc Natl Acad Sci USA 99, 3240-3245). Interestingly, NPY/AgRP neurons have projections to MCH-expressing neurons in the LHA (Elias, C. F., Aschkenasi, C., Lee, C., Kelly, J., Ahima, R. S., Bjorbaek, C., Flier, J. S., Saper, C. B., and Elmquist, J. K. (1999). Leptin differentially regulates NPY and POMC neurons projecting to the lateral hypothalamic area. Neuron 23, 775-786). Consequently, NPY/AgRP neurons may modulate the dopaminergic system through MCH-expressing neurons; this circuit would represent a major component within the CNS network that regulates locomotory behaviour (Zhou, Q. Y., and Palmiter, R. D. (1995). Dopamine-deficient mice are severely hypoactive, adipsic, and aphagic. Cell 83, 1197-1209.). Recent evidence links activity of particular cellular subpopulations of the VMH to neuronal activity in the arcuate (Sternson, S. M., Shepherd, G. M., and Friedman, J. M. (2005). Topographic mapping of VMH->arcuate nucleus microcircuits and their reorganization by fasting. Nat Neurosci 8, 1356-1363).

It is therefore interesting to note that genetic disruption of VMH neurons due to mutations in the gene encoding steroidogenic factor-1 has revealed an important role of the VMH in locomotor activity (Majdic, G., Young, M., Gomez-Sanchez, E., Anderson, P., Szczepaniak, L. S., Dobbins, R. L., McGarry, J. D., and Parker, K. L. (2002). Knockout mice lacking steroidogenic factor 1 are a novel genetic model of hypothalamic obesity. Endocrinology 143, 607-614).

Together with the observation that NPY receptors are expressed on NPY/AgRP neurons this finding suggests an autoregulatory negative feedback loop involving NPY and Bsx in the control of locomotor activity. Bsx mutant females show with 100% penetrance the same lactation defect which was reported for s/s females carrying a mutation in tyrosine¹¹³⁸ of the leptin receptor that attenuates Stat3 signalling. Thus, a possible direct cross-talk of Bsx function and leptin signalling in NPY/AgRP neurons to control locomotor activity may operate. Considering that a yet not understood neuronal network compensatory mechanism exists in case of germline Npy loss, the importance of Bsx for NPY/AgRP neuron function and body weight regulation may even be underestimated in the experiments as performed in the context of the present invention.

Future studies on the regulation of Bsx activity and the transcriptional network underlying NPY/AgRP physiology may provide new insight into the mechanisms that are responsible both for adaptive hyperphagia as well as spontaneous physical activity and might open new avenues to control obesity.

The low expression of Npy and Agrp after fasting in Bsx deficient mice and in in Bsx^{ΔHD/ΔHD}, ob/ob double-mutant mice suggested that Bsx may directly regulate Npy and Agrp expression. Rat PC12 cells, which express Npy at low levels (Higuchi et al., 1988), do not express endogenous Bsx. The inventors therefore hypothesized that expression of Bsx in PC12 cells would stimulate Npy expression. Quantitative RT-PCR revealed a 50-fold induction of Npy in cells transfected to express Bsx, whereas no signal was detected in mock-transfected cells (FIG. 8A). To see if this induction is specific to Bsx or if other homeobox transcription factors could produce similar effects, the inventors used the closely related homeobox genes Nkx2.1/TTF-1, which are co-expressed during development in the hypothalamus, as a control. Overexpression of Nkx2.1 or another control, Pit-1, led to only a modest increase in Npy RNA of four or two-fold, respectively (FIG. 8A). Activation of Npy expression can also be mimicked by treating PC12 cells with the cAMP analog DibcAMP and further stimulated with PMA (Higuchi, H., Yang, H. Y., and Sabol, S. L. (1988). Rat neuropeptide Y precursor gene expression. mRNA structure, tissue distribution, and regulation by glucocorticoids, cyclic AMP, and phorbol ester. J Biol Chem 263, 6288-6295), consistent with reports implicating the transcription factor CREB in Npy regulation during starvation (Shimizu-Albergine, M., Ippolito, D. L., and Beavo, J. A. (2001). Downregulation of fasting-induced cAMP response element-mediated gene induction by leptin in neuropeptide Y neurons of the arcuate nucleus. J Neurosci 21, 1238-1246). The inventors repeated this experiment in PC12 cells and found that Npy expression was activated about the same level by DibcAMP/PMA treatment as in the Bsx transfection experiment. Unexpectedly, when the inventors stimulated Bsx-transfected PC12 cells with DibcAMP/PMA, the inventors observed a synergistic increase in Npy expression (FIG. 8A). Using reciprocal GST pull down experiments, the inventors found that Bsx and CREB physically interact (FIG. 8E). The Npy gene resides within a gene desert encompassing about 500 kb. To identify the regulatory sequences to which CREB and Bsx bind to regulate Npy expression region, the inventors took a bioinformatic comparative sequence analysis approach. Aligning the Npy locus of various mammalian species only one region approximately 50 kb upstream of the annotated Npy gene start site could be identified that is conserved and contained a conserved CREB and homeobox binding site next to each other (FIG. 8B). When the inventors tested this DNA fragment in front of a basal promoter driving luciferase the inventors could recapitulate the synergistic activation seen in PC12 cells of the Npy gene by Bsx and cAMP (FIG. 8C). Furthermore, chromatin immunprecipitation (ChIP) using a Bsx specific antibody on H2BeGFP positive cells sorted from wild type and Bsx mutant animals demonstrated occupancy of the predicted homeobox binding site by Bsx in vivo (FIG. 8D).

Analogously, it has been reported that FoxO1 translocates to the nucleus during fasting where it stimulates Agrp gene expression (Kitamura, T., Feng, Y., Kitamura, Y. I., Chua, S. C., Jr., Xu, A. W., Barsh, G. S., Rossetti, L., and Accili, D. (2006). Forkhead protein FoxO1 mediates Agrp-dependent effects of leptin on food intake. Nat Med 12, 534-540). This can be mimicked using a constitutive FoxO1-ADA expression vector. The inventors therefore transfected AtT20 cells with combinations of Bsx and FoxO1 expression vectors and found that Bsx and FoxO1 co-operate to induce Agrp expression (FIG. 8G). Close to the reported FoxO1 and Stat3 binding sites there are two highly conserved homeobox binding sites in mammals (FIG. 8H). Employing a similar approach as for the Npy element the inventors could demonstrate that these sites are occupied by Bsx in vivo and are able to act in concert with the neighbouring FoxO1 site in Agrp gene induction (FIG. 8 I, J).

Furthermore, using GST pull down experiments, the inventors could show that Bsx and FoxO1 physically interact (FIG. 8K). These results together indicate that Bsx can directly stimulate Npy and Agrp expression and is able to physically interact with the two transcription factors CREB and FoxO1 that have been implicated in the fasting dependent up-regulation of these neuropeptides.

In conclusion, Bsx is a novel important component involved in the regulation of energy homeostasis that by itself directly and as CREB cofactor ensures NPY expression in the Arc nucleus and ghrelin responsiveness of the NPY/AgRP neurons. In addition deletion of Bsx in the ob/ob, leptin deficient, background prevents obesity because expression of NPY is not being increased. Thus providing the inventors with a possibly new anti-obesity drug target and maybe even more targets acting in addition to NPY downstream Bsx. The current data support the hypothesis that Bsx activity is one important molecular determinant of calorically relevant locomotory behaviour. Finally, the homeobox in Bsx is highly conserved among Drosophila, C. elegans, fish and mammals (Jones, B., and McGinnis, W. (1993). A new Drosophila homeobox gene, bsh, is expressed in a subset of brain cells during embryogenesis. Development 117, 793-806). This raises the possibility that Bsx might be part of an evolutionary conserved system that controls food acquisition and body weight regulation.

The following figures, sequences and examples merely serve to illustrate the invention and should not be construed to restrict the scope of the invention to the particular embodiments of the invention described in the examples. All references cited in the text are hereby incorporated in their entirety by reference.

SEQ ID No 1 to 7 show Bsx polypeptides and polynucleotides as employed according to the present invention.

SEQ ID No 8 to 67 show the sequences of oligonucleotides as used in the examples according to the present invention.

SEQ ID No 68 to 76 show the sequences of regulatory regions 1, 2 and 3 in the vicinity of Bsx as identified according to the invention for human, mouse, and rat, respectively

### FIGURES

FIG. 1 shows that Bsx mutant animals weight slightly less then their littermates maybe due to the modest reduced food intake (F—H). However they also showed an increase in their fat mass (I). In addition, their energy expenditure was nearly unchanged (J).

FIG. 2 depicts that Bsx−/− ob/ob where less obese compared to the ob/ob, but they were still larger than wild type control mice.

FIG. 3 depicts that the fat mass of the Bsx−/− ob/ob is significantly reduced compared to the ob/ob as expected from the difference in the weight curves.

FIG. 4 depicts the sequences that form part of the invention. In SEQ ID No. 7, small letters are untranslated regions, dotted lines indicate additional intron-sequences.

FIG. 5 depicts that the locomotor activity was severely reduced during the light and dark phase to a level comparable what has been described for leptin-deficient (ob/ob) mice.

FIG. 6 depicts that locomotor activity was not improved in Bsx^{ΔHD/ΔHD}, ob/ob mice compared to ob/ob mice despite the significant observed reduction in body weight. These results demonstrate that locomotor activity can be genetically uncoupled from body weight control in mice.

FIG. 7 depicts that (A) GHRP-6 administration in wild type mice increases number of positive Fos immunoreative cells in the arcuate compared to saline controls. (B) Double immunostaining shows that Fos positive cells do also express Bsx. (C) Fos immunoreactivity after GHRP-6 administration is not induced in homozygous Bsx^{ΔHD/ΔHD} mice compared to wild type controls. (D) 35S-in situ hybridization detecting ghrelin receptor (Ghsr) expression. Ghsr probes were exposed for 70 days. (E) Reduced 24 h food intake stimulation upon ghrelin administration in Bsx mutant mice compared to control animals.

FIG. 8 shows the regulation of Npy and Agrp gene expression by Bsx. (A) Npy expression analysis using quantitative RT-PCR from PC-12 cells transiently transfected with Bsx, Pit1, Nkx2.1 expression vectors and/or treated with PMA and/or DibcAMP; data were normalized for TBP (**P<0.01) (B) Sequence of the Npy regulatory element containing the conserved Bsx and CREB binding sites (C) Activation of a heterologous promoter driven by the Npy regulatory element in PC12 cells (D) Chromatin immunoprecipitation using a Bsx specific antibody shows occupancy of Bsx on the predicted binding site in isolated H2BeGFP labelled hypothalamic cells from heterozygous but not Bsx mutant mice. (E) GST pull-down interaction assay with GST::Bsx and in vitro translated full length CREB protein, and GST::CREB with in vitro translated Bsx protein. (F) Model of Npy gene regulation. (G) Agrp expression analysis using quantitative RT-PCR from AtT20 cells transiently transfected with Bsx and FoxO1 expression vectors; data were normalized for TBP (**P<0.001). (H) Sequence of the Agrp regulatory element containing the conserved Bsx, FoxO1 and Stat3 binding sites (I) Activation of a heterologous promoter driven by the Agrp regulatory element in AtT20 cells (J) Chromatin immunoprecipitation using a Bsx specific antibody shows occupancy of Bsx on the predicted binding site in isolated H2BeGFP labelled hypothalamic cells from heterozygous but not Bsx mutant mice. (K) GST pull-down interaction assay with GST::Bsx and in vitro translated full length FoxO1 protein and GST::FoxO1 with in vitro translated Bsx protein (L) Model of Agrp gene regulation.

### EXAMPLES

The inventors identified the brain-specific homeobox transcription factor Bsx in a screen for novel transcriptional regulators in the hypothalamic-pituitary axis. Bsx is expressed from e 10.5 in the ventral diencephalon which will give rise to the hypothalamus. In the adult mouse brain Bsx expression restricts to the arcuate nucleus and dorsomedial hypothalamus (DMH) with scattered expression in the lateral hypothalamus (LHA).

Immunofluorescence staining of adult brain sections with Bsx antibodies faithfully recapitulated the Bsx expression pattern obtained by in situ hybridization. Subsequent colocalization studies revealed that all NPY/AgRP neurons but no POMC neurons of the arcuate nucleus express Bsx. To assess the role of Bsx in mouse development the inventors generated two mouse strains lacking Bsx function. The Bsx^{ΔHD} allele has deleted the major part of the homeodomain which should result in a truncated protein unable to bind to DNA. The Bsx^{H2BEGFP} allele replaces the Bsx coding sequence in exon 1 with a sequence coding for the Histone2BEGFP fusion protein followed by a rabbit polyA signal which should also result in the absence of any functional Bsx protein.

The inventors obtained homozygous mutant adult Bsx^{ΔHD} or Bsx^{H2BEGFP} mice or mice double heterozygous for both alleles at the expected mendelian ratio. With a rat polyclonal antiserum generated against the C-terminus of Bsx the inventors no longer obtained any signal for Bsx on adult homozygous mutant brains demonstrating that the targeting strategy was successful. In situ/immuno doublefluoresence labeling with Npy or AgRP further demonstrated that the H2BEGFP expression pattern in Bsx^{H2BEGFP} mice faithfully reflected the endogenous Bsx expression. Next the inventors asked if the deletion of Bsx, due to its expression during embryonic development, leads to the loss of Bsx expressing neurons in adult homozygous mutant brains.

The inventors counted H2BEGF positive neurons throughout the arcuate nucleus and DMH of heterozygous Bsx^{H2BEGFP} and transheterozygous Bsx^{ΔHD}/Bsx^{H2BEGFP} mouse brains to find no difference in cell number, demonstrating that the loss of Bsx function does not lead to a loss of Bsx expressing neurons. Although mice homozygous mutant for the various Bsx alleles did not show any obvious phenotype and were fertile, the inventors wondered if loss of Bsx leads to a change in the gene expression program in these neurons. In situ hybridization for Npy revealed a strong down regulation of Npy expression specifically in the arcuate nucleus in Bsx mutant mice. Similarly immunofluorescence staining for NPY revealed a strong reduction of NPY staining in the arcuate nucleus. However, NPY staining pattern in the PVN and other hypothalamic region, although much weaker, appeared unchanged, suggesting that projection of NPY expressing neurons were not altered. AgRP was like Npy also downregulated in Bsx mutant brains. In contrast expression of POMC and cocaine-amphetamine regulated transcript (CART) were unchanged. The strong downregulation of Npy and AgRP in Bsx mutant brains prompted the inventors to perform a more detailed phenotypic analysis of Bsx mutant animals.

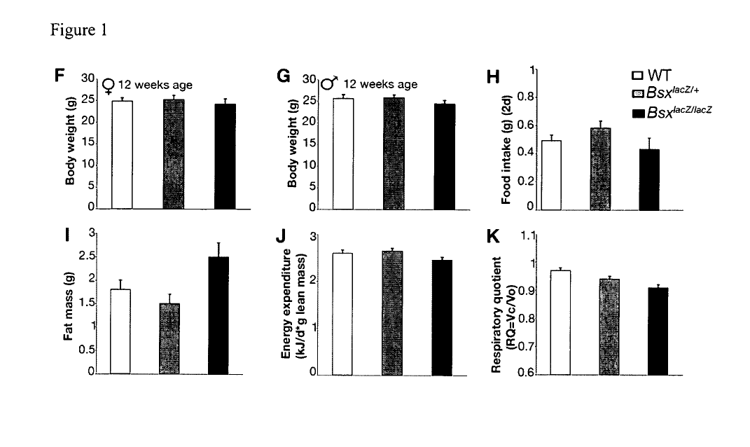

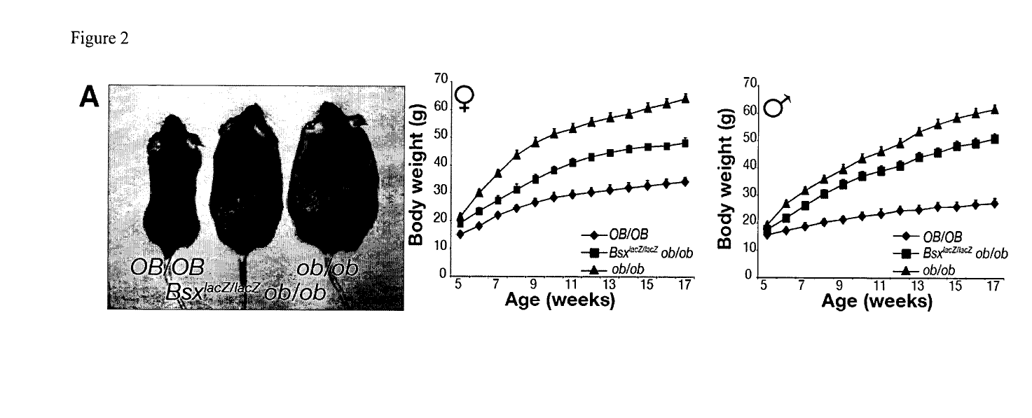

Bsx mutant animals weight slightly less then their littermates maybe due to the modest reduced food intake (FIGS. 1 F-H). However they also showed a significant increase in fat mass.

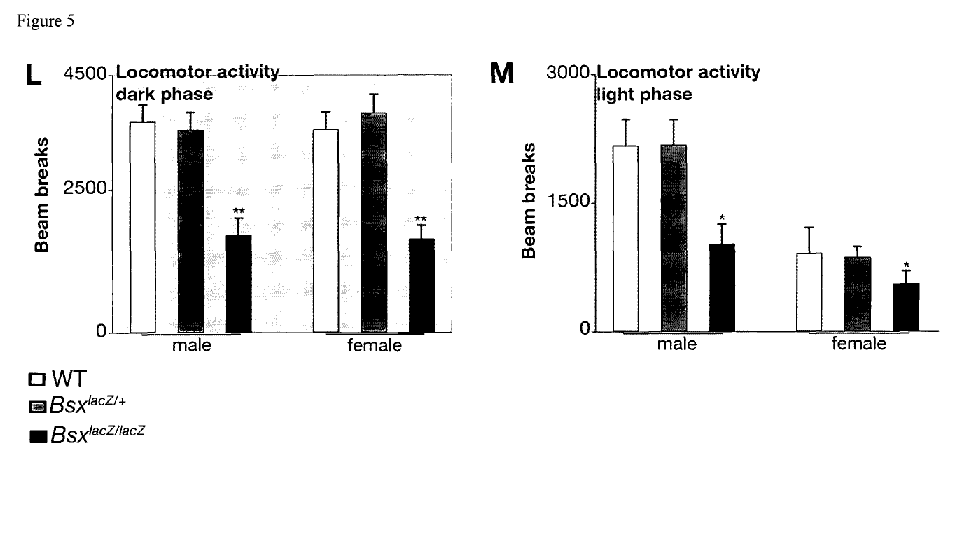

In addition, their energy expenditure was nearly unchanged (FIG. 1 J). Despite the strong downregulation of the two orexigenic peptides NPY and AgRP in Bsx mutant mice the observed modest change in physiological parameters are not surprising as even mice completely devoid of Npy and AgRP expression do not show a more severe phenotype. Surprisingly, locomotor activity in Bsx mutant mice was completely blunted during the night comparable to what has been described for leptin deficient (ob/ob) and leptin receptor deficient (db/db) animals (FIG. 5). However, locomotor activity is not impaired in Npy and/or Agrp deficient mice demonstrating that the defect in locomotor activity in Bsx mutant animals can not be explained by the down regulation of Npy and/or Agrp itself. In this respect it is interesting to note that reactivation of leptin receptor expression only in the arcuate nucleus nearly fully restores locomotor activity in leptin receptor deficient mice. As leptin receptor is expressed in NPY/AgRP and POMC neurons of the arcuate nucleus but Bsx only in NPY/AgRP neurons, the inventors suggest that Bsx function in NPY/AgRP neurons is required for locomotor activity. The decrease in locomotor activity may also be the reason for the observed small increase in fat mass of Bsx mutant mice. Due to the locomotor defect the inventors next asked if leptin-dependent STAT3 signalling is defective in NPY/AgRP neurons lacking Bsx function. Staining for phosho-Stat3 immunoreactivity after leptin injection did not reveal any difference between Bsx mutant and littermate controls in the number of phospho-Stat3 positive neurons in the arcuate nucleus. In addition, the activation of SOCS3, a direct transcriptional target of the leptin pathway, occurred suggesting that the leptin receptor/Stat3 signalling pathway is not impaired in Bsx mutant NPY/AgRP neurons.

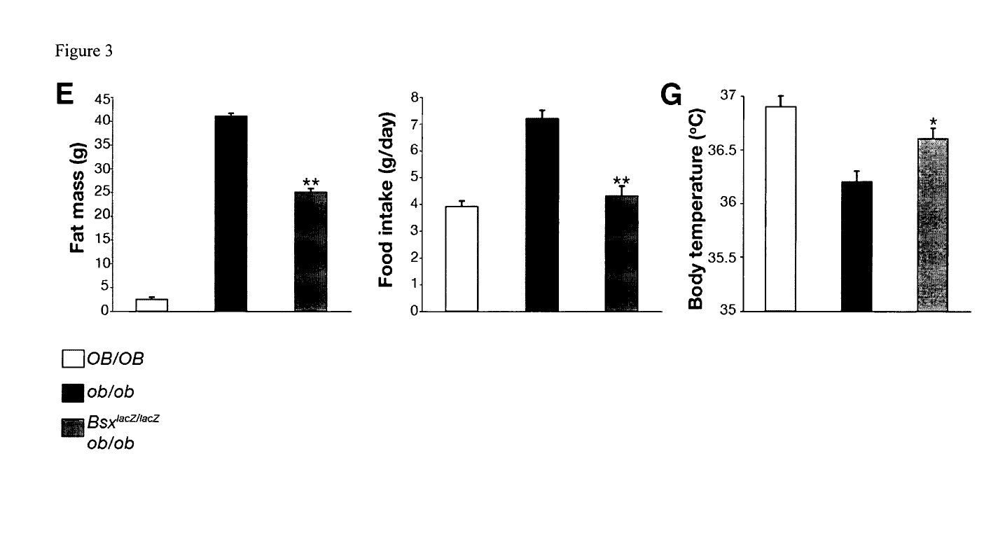

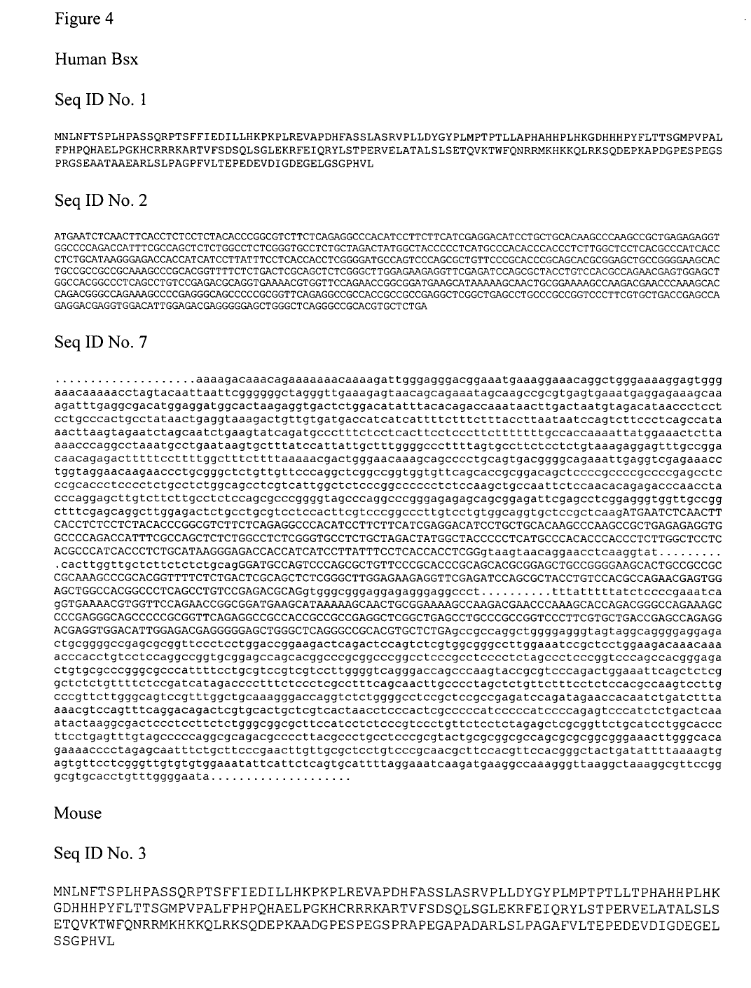

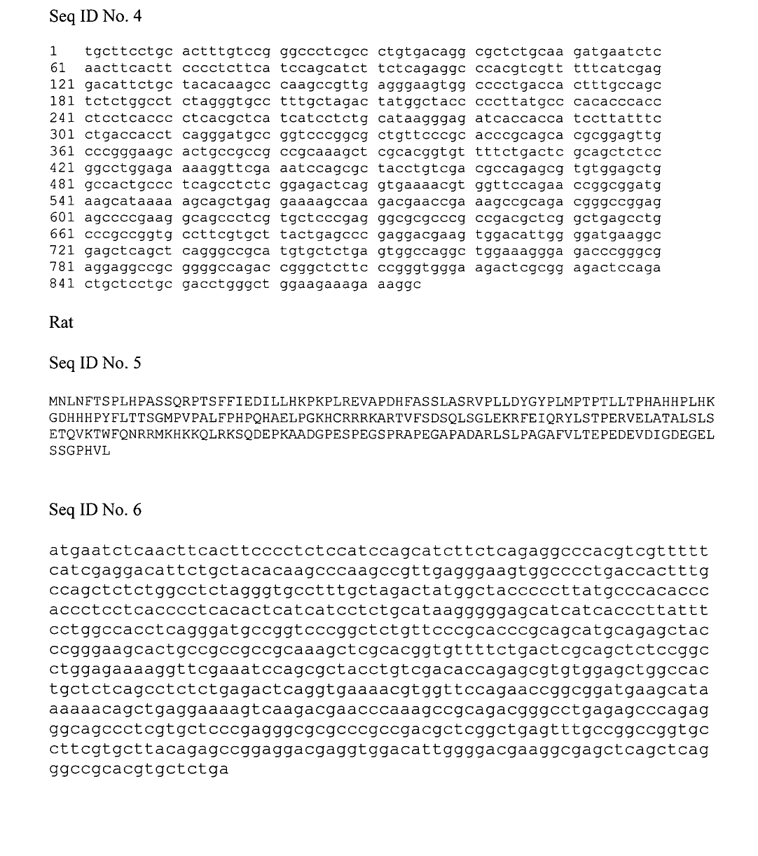

Bsx−/− ob/ob where less obese compared to the ob/ob, but they were still larger than wild type control mice (FIG. 2). The weight curves of both males and females showed that the Bsx−/− ob/ob double mutants weighed significantly less than the ob/ob, and more than wild type control, similar to the NPY−/− ob/ob. The difference between the body weight of Bsx−/− ob/ob and ob/ob was more pronounced after 9 weeks of age in females and after 7 weeks of age in males. NPY expression in the Arc of the Bsx−/− ob/ob was abolished compared to both basal levels of expression in the wild type control and of the ob/ob where NPY expression levels are constitutively strongly upregulated. Conversely POMC mRNA levels seemed to be equally repressed in both the ob/ob and the Bsx−/− ob/ob compared to wild type control. These results indicate that Bsx mediates NPY transcriptional induction in the ob/ob mutant but doesn't regulate transcription of POMC. In addition, fat mass of the Bsx−/− ob/ob is significantly reduced compared to the ob/ob as expected from the difference in the weight curves (FIG. 3E). To examine whether reduction of food intake, increase of energy expenditure, or both are responsible for rescuing the obesity syndrome of the compound Bsx−/− ob/ob the inventors performed measurements of food intake, body temperature, energy expenditure and respiratory quotient over 2 days and compared them to ob/ob.

The inventors measured for the double mutant Bsx−/− ob/ob 70% repression of the hyperphagia, 70% increase of energy expenditure, and increase of the body core temperature of more than half 0 C., reflecting a significant increase of O2 consumption and of the metabolic rate. This suggests that the suppression of the ob/ob severe obesity in the Bsx−/− ob/ob is due to the partial rescue of both factors that define body weight and fat mass, food consumption and energy expenditure, identical to NPY−/− ob/ob. Thus the Bsx−/− ob/ob mimics the phenotype of NPY−/− ob/ob. These results suggest that Bsx genetically interacts with leptin signalling pathway in the NPY/AgRP and targets NPY expression in these neurons.

Several studies have shown that NPY/AgRP neurons not only respond to leptin but also other endocrine hormones. In particular, ghrelin a gut derived peptide hormone has been shown to directly stimulate NPY/AgRP neurons. Injection of GHRP-6, a ghrelin mimetic, demonstrated that Bsx positive neurons in the arcuate nucleus become activated using Fos immunoreactivity as a read out. However, when the inventors injected GHRP-6 in Bsx mutant animals the inventors did not see any increase of FOS immunoreactivity in the arcuate nucleus suggesting that Bsx mutant NPY/AgRP neurons are unable to respond to ghrelin. However, in situ hybridization demonstrated that the expression of the growth hormone secretagogue receptor GHS-R which binds ghrelin is unchanged in the arcuate nucleus pointing to a cell-autonomous defect in NPY/AgRP neurons downstream of the receptor. The data presented so far have revealed two opposing defects in Bsx mutant NPY/AgRP neurons with respect to energy metabolism. Both, downregulation of Npy and AgRP and the non-responsiveness to ghrelin in Bsx mutant neurons should lead to leaner animals due to less food consumption and higher energy expenditure whereas the locomotor defect in Bsx mutant mice should decrease energy expenditure.

The severe locomotor defect in the absence of any significant weight change in Bsx mutant mice allowed the inventors to access the contribution of physical activity to body weight control. The inventors therefore wondered if Bsx mice are more susceptible to diet induced obesity.

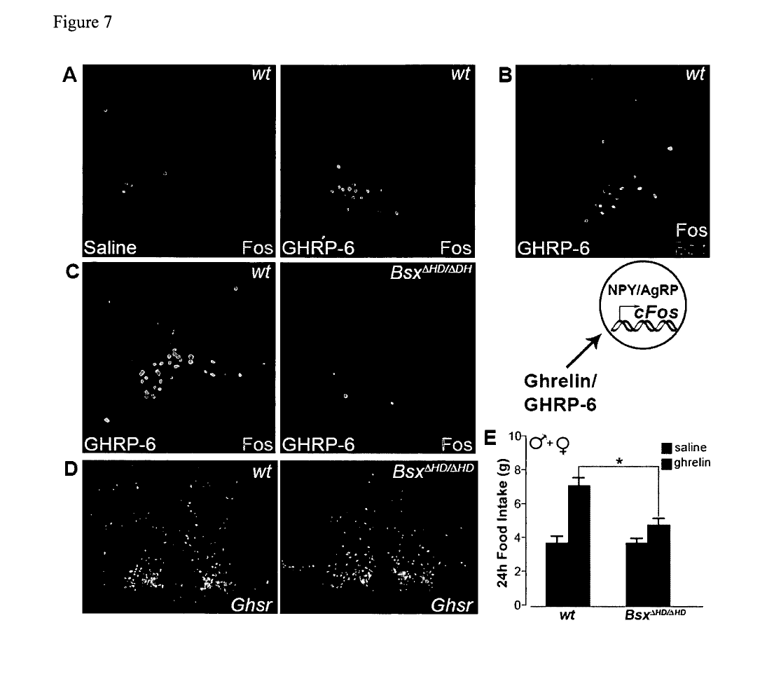

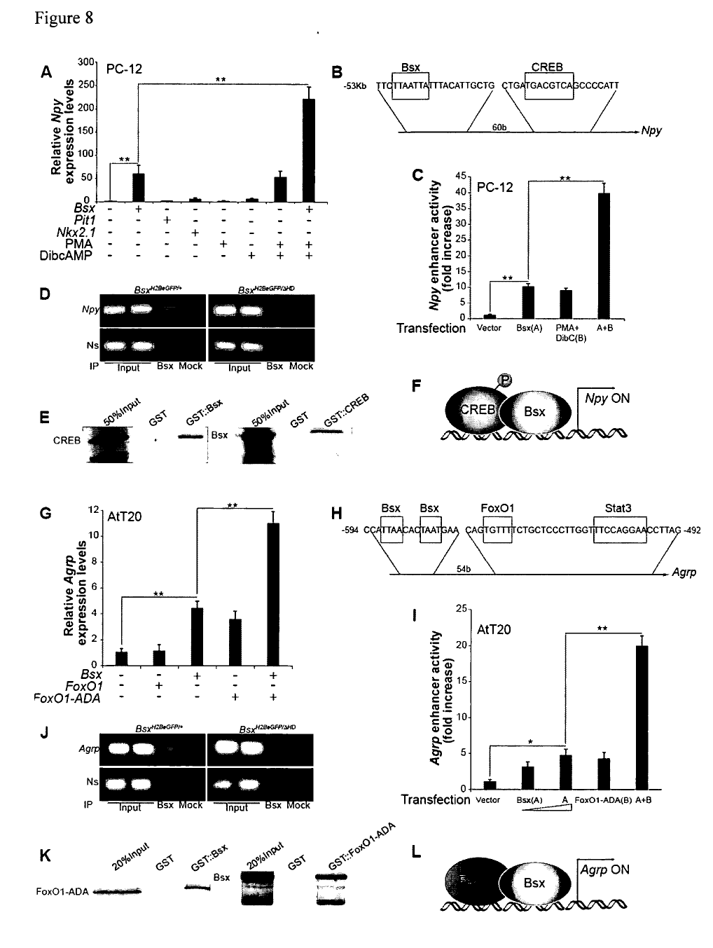

Ghrelin, a gut-derived peptide hormone whose circulating levels increase during fasting, is the only circulating factor that directly stimulates NPY/AgRP neurons (Cowley, M. A., Smith, R. G., Diano, S., Tschop, M., Pronchuk, N., Grove, K. L., Strasburger, C. J., Bidlingmaier, M., Esterman, M., Heiman, M. L., et al. (2003). The distribution and mechanism of action of ghrelin in the CNS demonstrates a novel hypothalamic circuit regulating energy homeostasis. Neuron 37, 649-661; Hewson, A. K., and Dickson, S. L. (2000). Systemic administration of ghrelin induces Fos and Egr-1 proteins in the hypothalamic arcuate nucleus of fasted and fed rats. J Neuroendocrinol 12, 1047-1049; Tschop, M., Smiley, D. L., and Heiman, M. L. (2000). Ghrelin induces adiposity in rodents. Nature 407, 908-913). Ghrelin levels increase with fasting, are thought to trigger food intake and have been implicated in the modulation of locomotor activity (Tang-Christensen, M., Vrang, N., Ortmann, S., Bidlingmaier, M., Horvath, T. L., and Tschöp, M. (2004). Central Administration of Ghrelin and Agouti-Related Protein (83-132) Increases Food Intake and Decreases Spontaneous Locomotor Activity in Rats. Endocrinology 145, 104645-104652; Wortley, K. E., Anderson, K. D., Yasenchak, J., Murphy, A., Valenzuela, D., Diano, S., Yancopoulos, G. D., Wiegand, S. J., and Sleeman, M. W. (2005). Agouti-related protein-deficient mice display an age-related lean phenotype. Cell Metab 2, 421-427). Injection of GHRP-6, a ghrelin mimetic, increased Fos immunoreactivity in arcuate Bsx-positive neurons (FIGS. 7A, B) in control but not in Bsx-mutant mice (FIG. 7C), suggesting that Bsx-deficient NPY/AgRP neurons are unable to respond to ghrelin. Injection of ghrelin has been shown to stimulate food intake (Tschop et al., 2000). Consistent with the blunted ghrelin-response of Bsxdeficient AgRP/NPY neurons, food intake upon ghrelin administration was significantly lower in Bsx mutant compared to control animals (FIG. 7E). In situ hybridization demonstrated that the ghrelin receptor, (growth hormone secretagogue receptor, Ghsr), is expressed normally in Bsx-deficient NPY/AgRP neurons, implying a blockade in ghrelin signalling downstream of the receptor (FIG. 7D) and a role for Bsx in these neurons that goes beyond the transcriptional regulation of Npy/Agrp.

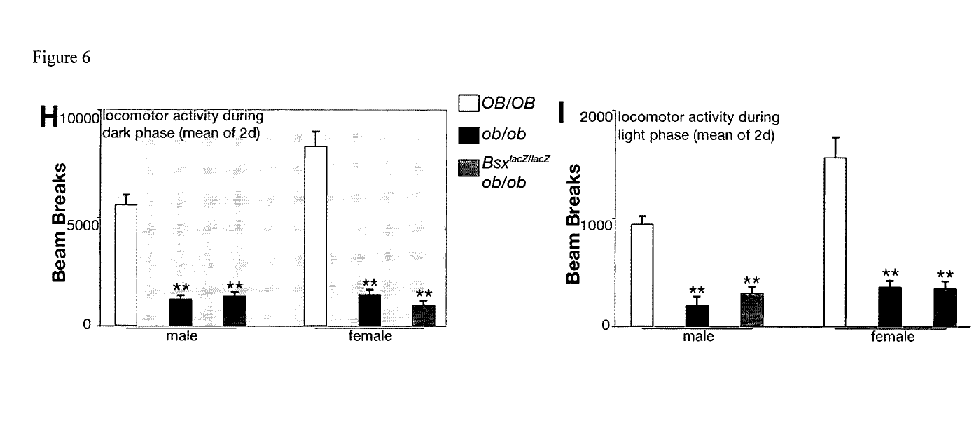

To separate the two opposing effects from each other the inventors took advantage of the fact that leptin deficient ob/ob mice display the same locomotor deficiency as Bsx mutant mice. However, ob/ob mice also show severe obesity due to the impairment of other physiological functions which allowed the inventors to directly evaluate the beneficial effects of Bsx loss with respect to energy homeostasis. The inventors therefore generated mice mutant for both, leptin and Bsx, to find that double homozygous mutant Bsx^{-/-}, ob/ob mice are visible leaner then their ob/ob littermates. This effect was more pronounced in females then males and was mainly due to a reduction in white fat mass. In situ hybridization analysis for Npy and AgRP in double homozygous mutant Bsx^{-/-}, ob/ob mice showed that instead of a strong derepression of Npy and AgRP which is normally observed in ob/ob animals, expression of the two neuropeptides did not exceed the low levels described in Bsx mutant animals. In contrast, no difference could be detected in POMC expression between ob/ob and Bsx^{-/-}, ob/ob mice which was in both cases lower then in wild type controls as previously reported. Consistent with the observed down-regulation of Npy expression in Bsx^{-/-}, ob/ob mice, food intake was only increased by 20% compared to control littermates, whereas ob/ob mice ate 70% more corresponding to a 60% reduction of hyperphagia. In addition, body temperature in Bsx^{-/-}, ob/ob mice was elevated compared to ob/ob mice contributing to enhanced energy expenditure maybe resulting from down-regulation of AgRP (FIG. 3 G). Because glucose homeostasis and glycaemic control is improved in ob/ob mice also deficient for Npy (ob/ob, Npy^{-/-}), the inventors measured these endocrine parameters in the Bsx^{-/-}, ob/ob animals. Bsx loss significantly ameliorates the glucosoria, hyperglycemia and increased serum insulin levels of ob/ob animals as well as the fertility of ob/ob females. Surprisingly, locomotor activity was not at all improved in Bsx^{-/-}, ob/ob mice compared to ob/ob mice despite the significant loss in body weight observed (FIG. 6).

The low expression of Npy in Bsx^{-/-}, ob/ob mice, in the absence of the inhibitory leptin signal, suggested a direct regulation of Npy by Bsx. To test this hypothesis the inventors wondered if certain neuronal-like cell lines including rat PC12 cells which express low levels of Npy also express Bsx. This was not the case for PC12 cells. The inventors therefore asked if expression of Bsx in PC12 cells could stimulate Npy expression. When the inventors expressed Bsx in PC12 cells and performed northern blot analysis the inventors could easily detect a signal for Npy whereas no signal was detected in mock transfected cells. When the inventors quantified the induction of Npy by Bsx using quantitative RT-PCR the inventors found a 50-fold induction. To see if this induction is specific to Bsx or if any other homeobox transcription factor could also induce Npy expression in PC12 cells the inventors used one of the closest related homeobox genes Nkx2.1/TTF-1 which is even co-expressed during development in the hypothalamus as a control. However, overexpression of Nkx2.1 or another control, Pit-1, lead only to a modest increase of four and two-fold, respectively demonstrating the specificity of Bsx for Npy expression. To evaluate the specificity of Bsx for Npy expression further, the inventors performed DNA chip analysis using the GE/Codelink whole genome rat chip to find that Npy is induced 70-fold on this platform whereas nearly all other genes are either not regulated at all or within the 2-fold range. In summary these data suggest that Bsx directly regulates Npy expression.

Several studies have shown that when mice are starved Npy expression is upregulated.

During starvation the transcription factor CREB becomes phosphorylated on Ser 33 in NPY/AgRP neurons which is a prerequisite for its full transcriptional activity. This activation of Npy expression can be mimicked by treating PC12 cells with a cAMP analog DibcAMP and further stimulated with PMA. The inventors repeated this experiment in PC12 cells to find that Npy expression was activated about the same level by DibcAMP/PMA treatment as in the Bsx transfection experiment. Unexpectedly, when the inventors stimulated Bsx transfected PC12 cells with DibcAMP/PMA the inventors observed a synergistic activation of Npy expression. Using reciprocal GST pull down experiments the inventors could show that Bsx and CREB physically interact and that this interaction is mediated via the c-terminal part of Bsx. The inventors starved Bsx mutant mice to ask if Npy expression under these conditions is also impaired. In case of wild type mice the inventors saw as reported a robust induction of Npy expression compared to fed animals. In contrast Npy expression in starved Bsx mutant mice remained much lower then fed control mice although a slight induction was seen consistent with the idea that CREB activity becomes stimulated under these conditions.

This result further demonstrates the requirement for Bsx for full Npy expression which can not be bypassed by either leptin deficiency or starvation.

### Materials and Methods

### Animals

Mice were housed in specific pathogen-free, light (12 hour light/dark cycle), temperature (23° C.) and humidity (50%-60% relative humidity) controlled conditions. Animals were fed with regular chow diet (Harlan Winkelmann, Teklad 2018S), for the high fat diet experiment; food and water were available ad libidum. The procedures for performing animal experiments were in accordance with the principles and guidelines of the LAR/EMBL. The Bsx^{ΔHD} allele was backcrossed for at least 10 generations to C57/B16J before the physiological measurements and interbreeding with ob mice were performed. The BsxH2beGFP allele is on a mixed 129Sv/C57B16J background. Mice carrying the ob mutation were obtained from Jackson Laboratories/Maine, USA.

### Generation of Bsx^{-/-} Mice—Genotyping Strategy

Bsx^{ΔHD} and Bsx^{H2beGFP} allele were generated using standard mouse embryonic stem cell technology. 129BAC was isolated and a 8 kb BamHI-fragment was subcloned containing the Bsx genomic locus. The flanking arms of the Bsx^{ΔHD} targeting vector were generated by PCR using the 8 kb genomic BamHI fragment in pBluescript SK (Stratagene) and the following primers:

5′arm:
(SEQ ID No 8)
5′-TGAAGCTTGGTGGCAGATTGAGTTCAAGAC-3′,
and
(SEQ ID No 9)
5′-GCGGATCCCGGTGCGGGAACAGCGCCGGGACCGG-3′
3′arm:
(SEQ ID No 10)
5′-GCGGATCCGGATTCTGCGTCCTGCTCTTCC-3′
and
(SEQ ID No 11)
5′-ATGCGGCCGCATAGCCCCAGACACTTGGTTCC-3′.

The 5′ arm was fused in frame with a lacZ-Neo cassette and a DTA cassette was added to the 3′ arm for negative selection. Unfortunately, the lacZ expression did shut down after the second generation.

Bsx^{ΔHD} allele was genotyped using the following primers:

Bsx4+Bsx6=350 bp wild type band; Bsx4+Bsx3=450 bp mutant band

Bsx3 (b-gal 3′):
(SEQ ID No 12)
5′-GAT GGG CGC ATC GTA ACC GTG CAT CTG CC-3′
Bsx4 (wt 5′):
(SEQ ID No 13)
5′-GAG AGA TGG GTT CCA GGA GCT TTG GAG C-3′
Bsx6 (wt 3′):
(SEQ ID No 14)
5′-GAA GAG CAG GAC GCA GAA TCC GGG GCA TCC-3′

Primers for Southern probe generation:

Bsx 5′ probe 5′:
(SEQ ID No 15)
5′-GGA CTA CAC GGG CAC TGT ACA GTT C-3′
Bsx 5′ probe 3′:
(SEQ ID No 16)
5′-GGA TTC TTG ATC TTC CCA AAC TCT GG-3′

The flanking arms of the BsxH2beGFP targeting vector were generated by PCR using the 8 kb genomic BamHI fragment in BSSK and the following primers:

5′arm
(SEQ ID No 17)
5′-CCAAGCTTGCAGAGCGCCTGTCACAGG-3′
and T7 primer;
3′arm
(SEQ ID No 18)
5′-CCTTAATTAACTCCTCACCCCTCACGCTC-3′
and T3 primer.

The 5′ arm was fused at the ATG in frame with a Histone2BeGFP-Neo cassette and a DTA cassette was added to the 3′ arm for negative selection.

Bsx^{H2beGFP} allele was genotyped using the following primers:

BsxHisWT5′:
(SEQ ID No 19)
5′-GCAGCTGCAGGCTCTTGAGTAGGC-3′
BsxHisWT3′:
(SEQ ID No 20)
5′-CTCTGAGAAGATGCTGGATGAAGAGG-3′
BsxHisMut3′:
(SEQ ID No 21)
5′-GGAAGCCTCACCTGCGATGCGCTCG-3′

In Situ Hybridization—Combined In Situ Hybridization—Immunohistochemistry

Anesthetized animals were transcardially perfused with 10% formalin; the brains were removed and postfixed over night at 4° C. with 10% formalin. Hybridization with α-³⁵S-UTP labelled (Amersham Bioscience) antisense RNA probes was performed as previously described (Ref) on 18 μm cryosections. Primers used for subcloning of the probes in pBluescript SK (Stratagene) were for the

Bsx:
(SEQ ID No 22)
ACCATGAATCTCAACTTCACTTCCCCTC
and
(SEQ ID No 23)
TCAGAGCACATGCGGCCCTGAGC;
Npy:
(SEQ ID No 24)
TGCTAGGTAACAAGCGAATGGG
and
(SEQ ID No 25)
AGTCCAGCCTAGTGGTGGCA,
Agrp:
(SEQ ID No 26)
ATGCTGACTGCAARGTTGCTGAG
and
(SEQ ID No 27)
TGCGACTACAGAGGTTCGTGG,

Pomc1 probe is as previously described (Treier, M., Gleiberman, A. S., O'Connell, S. M., Szeto, D. P., McMahon, J. A., McMahon, A. P., and Rosenfeld, M. G. (1998). Multistep signaling requirements for pituitary organogenesis in vivo. Genes Dev 12, 1691-1704).

Cart:
(SEQ ID No 28)
TGCTACCTTTGCTGGGTGC
and
(SEQ ID No 29)
TCACACAGCTTCCCGATCC;
Ghsr:
(SEQ ID No 30)
GCAACATGTGGAACGCGA
and
(SEQ ID No 31)
AAGCAGATGGCGAAGTAGCG;
Pmch:
(SEQ ID No 32)
TTCAGCTTCCAAGTCCATAAGGA
and
(SEQ ID No 33)
AGGTATCAAACTTGCCAACATGG;
Hcrt:
(SEQ ID No 34)
TTCTACAAAGGTTCCCTGGGC
and
(SEQ ID No 35)
ACCAAGAGACTGACAGCGGC;
Gal:
(SEQ ID No 36)
TTGATCCTGCACTGACCAGC
and
(SEQ ID No 37)
GATTGGCTTGAGGAGTTGGC;
Trh:
(SEQ ID No 38)
TGCGACTCCAAGATGCAGG
and
(SEQ ID No 39)
GGCATTAAGCCACCCTCCTCT;
Ghrh:
(SEQ ID No 40)
GGCTCCCACAACATCACAG
and
(SEQ ID No 41)
AGCTGAAGCAGAAGTAACAGGG.

For the transcription reaction the plasmids where digested with XbaI (NEB) and transcribed by T7 promoter. Hybridization signal was detected by autoradiography using Kodak NTB-2 liquid emulsion, the tissue was subsequently counterstained with Bis-Benzimide (Sigma). Autoradiographic exposure was for 7 days, 21 days, or 70 days, depending of the gene. Images were captured by using a Leica DC 500 camera attached to a Zeiss axiophot compound microscope.

For the fluorescent in situ hybridization, antisense RNA probes were labelled with digoxigenin-UTP (Roche Diagnostics) according to the manufacturer's protocol. Immunological detection of digoxigenin-labeled probes was performed according to manufacturer's protocol (Roche Diagnostics). After colour development with Fast Red tablets (Roche Diagnostic) the reaction was stopped and the sections were mounted with Mowiol for microscopy. Immunological detection of GFP was performed with a primary rabbit antibody against GFP (1:400 dilution, Torey Pines) incubated simultaneously with the anti-digoxigenin antibody and a secondary FITC coupled antibody (1:400 dilution, Molecular Probes) incubated after developing the in situ hybridization with the Fast Red tablets. Sections when next washed 3 times in PBS containing 1% Triton-X and mounted with Mowiol for microscopy. Images were captured by using a Leica TCS SP2 confocal Laser scanning microscope.

Measurement of Neuropeptide mRNA Levels

Bsx mutant and littermate controls were euthanized by cervical dislocation and RNA was extracted from hypothalamic wedges using Oiagen RNAeasy kit.

Relative mRNA expression was determined by quantitative RT-PCR on an ABI7500 instrument. Data analysis was done using relative expression software tool, REST (Pfaffl et al., 2002) using Gapdh and beta-actin as the reference genes. Oligonucleotide primers:

Npy,
(SEQ ID No 42)
5′CTCCGCTCTGCGACACTACA3′
and
(SEQ ID No 43)
AATCAGTGTCTCAGGGCTGGA;
Agrp,
(SEQ ID No 44)
GCGGAGGTGCTAGATCCACA
and
(SEQ ID No 45)
AGGACTCGTGCAGCCTTACAC;
Pomc,
(SEQ ID No 46)
ACCTCACCACGGAGAGCAAC
and
(SEQ ID No 47)
GCGAGAGGTCGAGTTTGCA;
Cart,
(SEQ ID No 48)
CCCGAGCCCTGGACATCTA
and
(SEQ ID No 49)
GCTTCGATCTGCAACATAGCG;
Gapdh,
(SEQ ID No 50)
TTCACCACCATGGAGAAGGC
and
(SEQ ID No 51)
CCCTTTTGGCTCCACCCT;
Actb,
(SEQ ID No 52)
CAACGAGCGGTTCCGATG
and
(SEQ ID No 53)
CACTGTGTTGGCATAGAGG.

### Immunohistochemistry

Anesthetized mice were perfused transcardially with PBS containing 2% paraformaldehyde. The brains were removed and postfixed over night in 2% paraformaldehyde. For double immunohistochemistry, free-floating 50 μm vibrotome sections were used (Leica VT1000S). Sections were next incubated in blocking solution (PBS containing 0.4% Triton-X, 5% serum corresponding to the secondary antibody) for 1 hour at room temperature followed by over night incubation with the primary antibodies at 4° C. The sections were washed the next day (0.4% Triton-X in PBS) and incubated for 1 hour at room temperature with the secondary antibodies. Bsx was detected with two antibodies the inventors generated, an antibody raised in rabbit immunized and boosted with the full length protein Bsx and diluted 1:200, and a second specific antibody raised in rat immunized and boosted with a truncated form of Bsx containing only the Carboxy-terminus of the protein diluted 1:100, NPY was detected with a rabbit anti-NPY (1:200 dilution, Peninsula Laboratories Inc, Bachem), AgRP with a rabbit anti-AgRP (1:200 dilution, Alpha Diagnostic international), β-endorphin with a rabbit anti-βendorphin (1:500 dilution, Parlow), α-MSH with a sheep anti-α-MSH (1:5000, Chemicon International), Fos with a rabbit anti-Fos (1:500, Oncogene Research products) and Phospho-Stat3 with a rabbit anti-Phospho-Stat3 (1:3000 dilution, Cell signalling technology). The secondary antibodies used were, Cy3-labeled anti-rabbit Immunoglobin G (IgG), Cy3-labelled anti-rat IgG, FITC-labelled anti-sheep, Cy2-labeled anti-rabbit IgG (all by Jackson ImmunoResearch raised in Donkey).

Colchicine, Leptin and Ghrelin Administration

For the colocalization immunohistochemistry performed for Bsx and the hypothalamic neuropeptides, wild type mice were treated with Colchicine (Sigma) in order to enhance cell body staining 24 hours before the perfusion. 60 μg Colchicine (10 mg/ml in 0.9% saline) has been administrated in the third Ventricule of the mice, by usage of a stereotaxical table.

8 to 10 week old wild-type and Bsx^{ΔHD/ΔHD} mice that were food deprived during 48 hours, were injected intravenously with 2 μg of leptin/gr of body weight (Parlow). Mice were anesthetized 45 minutes after the injections and perfused transcardially with PBS containing 2% paraformaldehyde; brains were removed and postfixed over night at 4° C. as described previously for immunohistochemistry.

For the GHRP-6 experiment, 8-10 week old wild type and Bsx^{ΔHD/ΔHD} were used. They were given intravenous injection of 0.5 μg GHRP-6/gr body weight (GHRP-6 synthetic peptide, Bachem). Mice were sacrificed as described above 90 minutes after the injections and brains were postfixed as mentioned above.

### Physiological Measurements

The Bsx^{ΔHD} allele was backcrossed for at least 10 generations to C57/B16J before the physiological measurements were performed.

### Body Composition

Body fat mass was measured in all mice on day 75 of age in duplicates using Nuclear Magnetic Resonance (QMR, EchoMRI, Quantitative Magnetic Resonance Body Composition Analyzer, Echo Medical Systems, Houston, Tex., USA (Taicher et al., 2003; Tinsley et al., 2004)), which allows repeated measurements in conscious animals. Lean mass was calculated by subtracting fat mass from body weight measured prior to NMR measurement.

### Locomotor Activity

Gross locomotor activity of mice was measured using biotelemtry (Mini Mitter Co., Inc., Bend, Oreg., USA). This system requires implantation of transponders into the abdominal cavity and the mouse cage to be placed on a receiver. The current location of the transponder signal on the receiver area compared to the previous measurement is interpreted as movement. At the age of 8 to 12 weeks mice were implanted with transponders under Ketamin (1 μl/g, Ketamin Gräub, A. Albrecht, Aulendorf)/xylazine hydrochloride (0.2 μl/g, Rompun, Bayer Vital, Leverkusen) anaesthesia. The abdominal cavity was closed using absorbable suture (PGA Resorba, Resorba, Nuremberg), the skin was closed with clips (Becton Dickinson) that were removed 1 week after surgery. Localization of the transponder signal on the receiver was measured every 5 minutes. A second technique for the measurement of mouse locomotor activity within home cage environment was based on number of breaks of an infrared light beam system (TSE, Bad Homburg) and was used for confirmation of principal findings as well as for the quantification of fasting induced stimulation of locomotor activity.

### Energy Expenditure

Energy expenditure was measured by indirect calorimetry using a self-constructed system equipped with the gas analyzing system Advance Optima from ABB AG (Mannheim, Germany, formerly Hartmann and Braun). The system provides one measurement every six minutes per cage. Energy expenditure of mice was evaluated at the age of 8-12 weeks. Mice were adapted to respiratory cages for two days. Adaptation was followed by a 2 days measurement period. Before and after the measurement period mice were analyzed for body composition using Nuclear Magnetic Resonance imaging for calculation of lean mass specific energy expenditure.

### Ghrelin Induced Food Intake

Ghrelin induced food intake was tested in a cross-over design, with every mouse receiving saline and ghrelin injection leaving a washout phase of 2 days inbetween injections. On treatment day, non-fasted mice were intraperitoneally injected with 500 μl of either saline (Sigma-Aldrich Company, Irvine, UK) or ghrelin (0.8 g/ml dissolved in saline) in the early light phase. Mice were then given free access to water and pre-weighed food presented in a customized feeder, which also allows for detection of food spillage, thereby providing accurate values for food consumption.

Feeders were weighed 1, 2, 4, 6, and 24 hours following injection for monitoring of ghrelin induced feeding. Ghrelin was synthesized and generously provided by Richard DiMarchi (Dept. of Chemistry, Indiana University, Bloomington, Ind., USA).

### GST Pull-Down Assay

Briefly after purification with glutathione sepharose beads, GST and GST fusion proteins were loaded on an SDS-acrylamide gel for quantification. 2 μg of protein coupled with the matrix were used in each sample. The full length mouse Bsx, CREB and Foxo1 proteins were expressed and purified as GST fusions proteins in the pET-41a-c bacteria vector (Novagen). GST::Bsx, GST::CREB and GST::FOXO1 were added to the standard GST pull-down with in vitro translated CREB, full length Bsx or FOXO1 labelled with Methionine-35S (in vitro translation kit: TNT coupled reticulocyte lysate systems, Promega, Methionine-35S from Amersham). The reaction was allowed to proceed for two hours at 40 C. in PBS 0.1% Tween w/w. Samples were next washed 5 times with PBS containing 0.5% Tween w/w and loaded on a SDS-acrylamide gel. The data were then analyzed by autoradiography.

### Cell Culture-qRT-PCR Analysis

AtT20 cells and PC-12 cells were obtained from ATCC and transfected with lipofectamin 2000 (Invitrogen). Cells were treated with Phrobol12-myristate-13-acetate (PMA, Sigma) and 2′-O-Dibutryladenosine 3′,5′ cyclic mopnophosphate sodium salt (DibcAMP, Sigma) as indicated. Quantitative real-time fluorescent-based reverse polymerase chain reaction (QRT-PCR) has been performed (SDS ABI Applied Biosystems and ABI SYBR green PCR master mix) in order to quantify Npy and Agrp mRNA isolated from PC-12 cells. For cDNA synthesis, 5 μg total RNA was reverse transcribed using the SuperScript first strand synthesis system for RT-PCR (Invitrogen) according to manufacturers instructions. A total of 100 ng total RNA was used per qRT-PCR in 25 μl containing 1×SYBR Green PCR Master mix and 300 nM each of the gene-specific primer pair.

### Npy Specific Primers:

(SEQ ID No 54)
5′-TCTCATCTCATCCTGTGAAACCAGTCTGC-3′ (forward)
and
(SEQ ID No 55)
5′-AAGGGAAATGGGTCGGAATCCAGCCTGG-3′(reverse).

Agrp Specific Primers:

(SEQ ID No 56)
5′-GCGCACAGGTCGCAGCAAGGTA-3′ (forward)
and
(SEQ ID No 57)
5′-GCAGAGGTGCTAGATCCACAGAA-3′ (reverse)

TATA Binding Protein (TBP) was used as a reference gene with the specific primers

(SEQ ID No 58)
5′-GCACAGGAGCCAAGAGTGA-′3/
(SEQ ID No 59)
5′-AGCTCCCCACCATGTTCTG-′3.

### ChIP Assays

‘Carrier’ ChIP assay was assayed on 25,000 FACS sorted GFP positives neurons from 12 BsxH2BeGFP/+ and 12 BsxH2BeGFP/DHD mice (O'Neill, L. P., VerMilyea, M. D., and Turner, B. M. (2006). Epigenetic characterization of the early embryo with a chromatin immunoprecipitation protocol applicable to small cell populations. Nat Genet 38, 835-841). GFP positive neurons were sorted on a modified Dako MoFlo sorter (DAKO GmbH, Hamburg Germany D-22083). The sorter was configured in the following way. 150 mW from a 488 nm argon Ion Laser (Coherent GmbH Germany) A beam splitter was placed in front on the laser in order to monitor sort deflections. Only approximately 60% of the light was used to illuminate the cells the rest was used in monitoring the sort deflections. Four detectors were used for light collection. The Forward Scatter (FSC) diode had a 488/10 Band Pass (BP) filter and a 0.6 neutral density (ND) filter. The Side Scatter (SSC) PMT (−6) had a 488/10 BP and a 1.0 ND. The GFP Fluorescence (FL-1) PMT (−15) had a 512/15BP and red Auto-fluorescence (FL-2) PMT (−12) had a 670/30BP. A 555 Long Pass dichroic filter was used to separate out the GFP fluorescence from the rest of the auto-fluorescence, FL-1 from FL-2. Two dot density plots were constructed; FSC Lin vs. SSC Log and FL-1 Log vs. FL-2 Log. GFP positive signals were identified from the Auto-Fluorescence, and back-gated onto the FSC vs. SSC plot to identify cells from debris. A gate was then used to remove debris from the fluorescence plot. The GFP particles were identified and a sort gate was drawn. These gates were combined along with the Purify-One sort mode of the sorter to give the sort criteria.

All PCR reactions were performed in duplicate with mouse and D. melanogaster DNA controls to monitor cross-hybridization. The primers used for the Agrp were:

(SEQ ID No 60)
5′-CGGAAGGGAGCAGCCAT-3′ (forward)
and
(SEQ ID No 61)
5′-TCCTGGCTCTCCCTCCT-3′ (reverse)
(−676 to −479)

and the unspecific ones: 5′-GCAGACAGCATCCAG-3′ (forward) (SEQ ID No 62) and 5′-CGATGGAACATCCAGT-3′ (reverse) (SEQ ID No 63) (−11.3 Kb to −11.4 Kb).

For the Npy: 5′-GCAGCCTTCATATCG-3′ (forward) (SEQ ID No 64) and 5′-GCTCTGTGATGTTC-3′ (reverse) (SEQ ID No 65) (−48.5 Kb to −48.3 Kb) and the unspecific are: 5′-GCATGGCTCACCAT-3′ (forward) (SEQ ID No 66) and 5′-CCAGCCAGCCCAGTA-3′ (reverse) (SEQ ID No 67) (−59.9 Kb to −59.7 Kb).

The rabbit antibody against Bsx was used for the IP and the rabbit antibody against GFP was used for the mock control IP.

### Biochemical and Hormonal Assays

The presence of glucose in urine (glucosoria) was tested with ComburTest (Roche). Glucose in serum was measured using the Reflotron system (Roche). Insulin was determined with EZRMI-13K from LINGO Research, USA.

### Statistical Analyses

Data are presented as means±s.e.m. All data were normally distributed. Two-sample t-test was applied to calculate significance between the fat mass of Bsx^{ΔHD/ΔHD} and the control wt. Analysis of variance was calculated by applying Kruskal-Wallis tests (for the re-feeding and locomotion measurements during the fasting experiment, for the real-time PCR data, the fat mass, food intake and body temperature measurements of the double mutant Bsx^{ΔHD/ΔHD} ob/ob mice and the endocrine parameters measurements). The test was followed by Tukey's post-hoc tests (for the real-time PCR data, the fat mass, food intake and body temperature measurements of the double mutant Bsx^{ΔHD/ΔHD} ob/ob mice and the endocrine parameters measurements). For all analyses, significance was assigned at the P<0.05 level.

1. An isolated protein comprising the same or substantially the same amino acid sequence of SEQ ID NO: 1, or a splice variant or a salt thereof.

2. The protein according to claim 1, which comprises at least one fragment of said amino acid sequence.

3. A nucleic acid, which comprises at least one nucleic acid encoding a protein according to claim 1.

4. The nucleic acid according to claim 3, which consists of DNA, CNA, PNA, or RNA.

5. The nucleic acid according to claim 4 comprising a nucleic acid of SEQ ID NO: 2.

6. The nucleic acid according to claim 3 further comprising at least one promoter, enhancer, intron and/or polyA-sequence.

7. A nucleic acid, which is complementary to the nucleic acid according to claim 3.

8. A vector comprising a protein comprising the same or substantially the same amino acid sequence of SEQ ID NO: 1, or a splice variant or a salt thereof and/or a nucleic acid according to claim 3.

9. The vector according to claim 8 selected from the group of vectors consisting of plasmids, phagemids, phages, cosmids, artificial mammalian chromosomes, knock-out or knock-in constructs, viruses, naked DNA, virosomes, liposomes, and nucleic acid coated particles.

10. An isolated host cell comprising a protein comprising the same or substantially the same amino acid sequence of SEQ ID NO: 1, or a splice variant or a salt thereof, and/or a nucleic acid according to claim 3.

11. The cell according to claim 10, which is a stem cell.

12. A transgenic non-human animal generated from a cell according to claim 10.

13. An antibody directed against a protein according to claim 1.

14. A method for producing a protein comprising the same or substantially the same amino acid sequence of SEQ ID NO: 1, or a splice variant or a salt thereof and/or a nucleic acid according to claim 3 comprising the steps of:
a) cultivating a cell comprising a protein comprising the same or substantially the same amino acid sequence of SEQ ID NO: 1, or a splice variant or a salt thereof, and/or a nucleic acid according to claim 3, and
b) isolating said protein and/or said nucleic acid.

15. A method of isolating a compound that is interacting with a protein comprising the same or substantially the same amino acid sequence of SEQ ID NO: 1, or a splice variant or a salt thereof, said method comprising the steps of:
a) contacting said protein with at least one potentially interacting compound, and
b) measuring binding of said compound to said protein.

16. The method according to claim 15, further comprising the steps of
a) selecting a binding compound,
b) modifying the binding compound, to generate a variety of modified binding compounds,
c) contacting said protein with each of the modified binding compounds,
d) measuring binding of said modified compounds to said protein, and
e) optionally, repeating steps a) to d) for one or more times.

17. The method according to claim 15, further comprising the step of:
measuring the biological function of a protein comprising the same or substantially the same amino acid sequence of SEQ ID NO: 1, or a splice variant or a salt thereof in the presence or absence of said binding compound.

18. The method according to claim 15, wherein contacting said protein with at least one potentially interacting compound is performed in the presence of cofactors, such as CREB and/or FoxO1.

19. The method according to claim 15, further comprising the step of admixing the interacting compound with suitable auxiliary substances and/or additives.

20. A pharmaceutical composition comprising a protein comprising the same or substantially the same amino acid sequence of SEQ ID NO: 1, or a splice variant or a salt thereof; a nucleic acid according to claim 3; a cell comprising a protein comprising the same or substantially the same amino acid sequence of SEQ ID NO: 1, or a splice variant or a salt thereof and/or a nucleic acid according to claim 3; an antibody directed against a protein comprising the same or substantially the same amino acid sequence of SEQ ID NO: 1, or a splice variant or a salt thereof; and/or a binding compound isolated by a method of isolating a compound that is interacting with a protein comprising the same or substantially the same amino acid sequence of SEQ ID NO: 1, or a splice variant or a salt thereof, said method comprising the steps of:
a) contacting said protein with at least one potentially interacting compound, and
b) measuring binding of said compound to said protein; and, optionally, suitable auxiliary substances and/or additives.

21-22. (canceled)

23. Use of a protein comprising the same or substantially the same amino acid sequence of SEQ ID NO: 1, or a splice variant or a salt thereof; or a nucleic acid according to claim 3; or an antibody directed against a protein comprising the same or substantially the same amino acid sequence of SEQ ID NO: 1, or a splice variant or a salt thereof; as a diagnostic marker for the diagnosis of a disease or disease state.

24. A method of preventing or treating obesity and/or related disease in a mammal in need thereof, comprising administering an effective amount of a pharmaceutical composition according to claim 20 to said mammal.


SEQUENCE LISTING (76 sequences)

SEQ ID NO: 1 - protein, 233 aa, Homo sapiens
MNLNFTSPLHPASSQRPTSFFIEDILLHKPKPLREVAPDHFASSLASRVPLLDYGYPLMPTPTLLAPHAHHPLHKGDHHHPYFLTTSGMPVPALFPHPQHAELPGKHCRRRKARTVFSDSQLSGLEKRFEIQRYLSTPERVELATALSLSETQVKTWFQNRRMKHKKQLRKSQDEPKAPDGPESPEGSPRGSEAATAAEARLSLPAGPFVLTEPEDEVDIGDEGELGSGPHVL

SEQ ID NO: 2 - DNA, 702 bp, Homo sapiens
atgaatctcaacttcacctctcctctacacccggcgtcttctcagaggcccacatccttcttcatcgaggacatcctgctgcacaagcccaagccgctgagagaggtggccccagaccatttcgccagctctctggcctctcgggtgcctctgctagactatggctaccccctcatgcccacacccaccctcttggctcctcacgcccatcaccctctgcataagggagaccaccatcatccttatttcctcaccacctcggggatgccagtcccagcgctgttcccgcacccgcagcacgcggagctgccggggaagcactgccgccgccgcaaagcccgcacggttttctctgactcgcagctctcgggcttggagaagaggttcgagatccagcgctacctgtccacgccagaacgagtggagctggccacggccctcagcctgtccgagacgcaggtgaaaacgtggttccagaaccggcggatgaagcataaaaagcaactgcggaaaagccaagacgaacccaaagcaccagacgggccagaaagccccgagggcagcccccgcggttcagaggccgccaccgccgccgaggctcggctgagcctgcccgccggtcccttcgtgctgaccgagccagaggacgaggtggacattggagacgagggggagctgggctcagggccgcacgtgctctga

SEQ ID NO: 3 - protein, 232 aa, Mus musculus
MNLNFTSPLHPASSQRPTSFFIEDILLHKPKPLREVAPDHFASSLASRVPLLDYGYPLMPTPTLLTPHAHHPLHKGDHHHPYFLTTSGMPVPALFPHPQHAELPGKHCRRRKARTVFSDSQLSGLEKRFEIQRYLSTPERVELATALSLSETQVKTWFQNRRMKHKKQLRKSQDEPKAADGPESPEGSPRAPEGAPADARLSLPAGAFVLTEPEDEVDIGDEGELSSGPHVL

SEQ ID NO: 4 - DNA, 875 bp, Mus musculus
tgcttcctgcactttgtccgggccctcgccctgtgacaggcgctctgcaagatgaatctcaacttcacttcccctcttcatccagcatcttctcagaggcccacgtcgtttttcatcgaggacattctgctacacaagcccaagccgttgagggaagtggcccctgaccactttgccagctctctggcctctagggtgcctttgctagactatggctacccccttatgcccacacccaccctcctcacccctcacgctcatcatcctctgcataagggagatcaccaccatccttatttcctgaccacctcagggatgccggtcccggcgctgttcccgcacccgcagcacgcggagttgcccgggaagcactgccgccgccgcaaagctcgcacggtgttttctgactcgcagctctccggcctggagaaaaggttcgaaatccagcgctacctgtcgacgccagagcgtgtggagctggccactgccctcagcctctcggagactcaggtgaaaacgtggttccagaaccggcggatgaagcataaaaagcagctgaggaaaagccaagacgaaccgaaagccgcagacgggccggagagccccgaaggcagccctcgtgctcccgagggcgcgcccgccgacgctcggctgagcctgcccgccggtgccttcgtgcttactgagcccgaggacgaagtggacattggggatgaaggcgagctcagctcagggccgcatgtgctctgagtggccaggctggaaagggagacccgggcgaggaggccgcggggccagaccgggctcttcccgggtgggaagactcgcggagactccagactgctcctgcgacctgggctggaagaaagaaaggc

SEQ ID NO: 5 - protein, 232 aa, Rattus norvegicus; identical to SEQ ID NO: 3

SEQ ID NO: 6 - DNA, 699 bp, Rattus norvegicus
atgaatctcaacttcacttcccctctccatccagcatcttctcagaggcccacgtcgtttttcatcgaggacattctgctacacaagcccaagccgttgagggaagtggcccctgaccactttgccagctctctggcctctagggtgcctttgctagactatggctacccccttatgcccacacccaccctcctcacccctcacactcatcatcctctgcataagggggagcatcatcacccttatttcctggccacctcagggatgccggtcccggctctgttcccgcacccgcagcatgcagagctacccgggaagcactgccgccgccgcaaagctcgcacggtgttttctgactcgcagctctccggcctggagaaaaggttcgaaatccagcgctacctgtcgacaccagagcgtgtggagctggccactgctctcagcctctctgagactcaggtgaaaacgtggttccagaaccggcggatgaagcataaaaaacagctgaggaaaagtcaagacgaacccaaagccgcagacgggcctgagagcccagagggcagccctcgtgctcccgagggcgcgcccgccgacgctcggctgagtttgccggccggtgccttcgtgcttacagagccggaggacgaggtggacattggggacgaaggcgagctcagctcagggccgcacgtgctctga

SEQ ID NO: 7 - DNA, 2802 bp, Homo sapiens
  5′ UTR (1)..(1000)
  Intron (1263)..(1313): Intron sequence shortened
aaaagacaaacagaaaaaaacaaaagattgggagggacggaaatgaaaggaaacaggctgggaaaaggagtgggaaacaaaaacctagtacaattaattcggggggctagggttgaaagagtaacagcagaaatagcaagccgcgtgagtgaaatgaggagaaagcaaagatttgaggcgacatggaggatggcactaagaggtgactctggacatatttacacagaccaaataacttgactaatgtagacataaccctcctcctgcccactgcctataactgaggtaaagactgttgtgatgaccatcatcattttctttctttaccttaataatccagtcttccctcagccataaacttaagtagaatctagcaatctgaagtatcagatgccctttctcctcacttcctcccttctttttttgccaccaaaattatggaaactcttaaaacccaggcctaaatgcctgaataagtgctttatccattattgctttggggcccttttagtgccttctcctctgtaaagaggagtttgccggacaacagagactttttccttttggctttcttttaaaaacgactgggaacaaagcagcccctgcagtgacggggcagaaattgaggtcgagaaacctggtaggaacaagaaccctgcgggctctgttgttcccaggctcggccggtggtgttcagcaccgcggacagctccccgccccgccccgagcctcccgcaccctcccctctgcctctggcagcctcgtcattggctctcccggcccccctctccaagctgccaattctccaacacagagacccaacctacccaggagcttgtcttcttgcctctccagcgcccggggtagcccaggcccgggagagagcagcggagattcgagcctcggagggtggttgccggctttcgagcaggcttggagactctgcctgcgtcctccacttcgtcccggcccttgtcctgtggcaggtgctccgctcaagatgaatctcaacttcacctctcctctacacccggcgtcttctcagaggcccacatccttcttcatcgaggacatcctgctgcacaagcccaagccgctgagagaggtggccccagaccatttcgccagctctctggcctctcgggtgcctctgctagactatggctaccccctcatgcccacacccaccctcttggctcctcacgcccatcaccctctgcataagggagaccaccatcatccttatttcctcaccacctcgggtaagtaacaggaacctcaaggtatcacttggttgctcttctctctgcagggatgccagtcccagcgctgttcccgcacccgcagcacgcggagctgccggggaagcactgccgccgccgcaaagcccgcacggttttctctgactcgcagctctcgggcttggagaagaggttcgagatccagcgctacctgtccacgccagaacgagtggagctggccacggccctcagcctgtccgagacgcaggtgggcgggaggagagggaggcccttttatttttatctccccgaaatcaggtgaaaacgtggttccagaaccggcggatgaagcataaaaagcaactgcggaaaagccaagacgaacccaaagcaccagacgggccagaaagccccgagggcagcccccgcggttcagaggccgccaccgccgccgaggctcggctgagcctgcccgccggtcccttcgtgctgaccgagccagaggacgaggtggacattggagacgagggggagctgggctcagggccgcacgtgctctgagccgccaggctggggagggtagtaggcaggggaggagactgcggggccgagcgcggttccctcctggaccggaagactcagactccagtctcgtggcgggccttggaaatccgctcctggaagacaaacaaaacccacctgtcctccaggccggtgcggagccagcacggcccgcggcccggcctcccgcctcccctctagccctcccggtcccagccacgggagactgtgcgcccgggcgcccattttcctgcgtccgtcgtccttggggtcagggaccagcccaagtaccgcgtcccagactggaaattcagctctcggctctctgttttctccgatcatagacccctttctccctcgcctttcagcaacttgcccctagctctgttctttcctctccacgccaagtccttgcccgttcttgggcagtccgtttggctgcaaagggaccaggtctctggggcctccgctccgccgagatccagatagaaccacaatctgatctttaaaacgtccagtttcaggacagactcgtgcactgctcgtcactaacctcccactcgcccccatcccccatccccagagtcccatctctgactcaaatactaaggcgactccctccttctctgggcggcgcttccatcctctcccgtccctgttctcctctagagctcgcggttctgcatcctggcacccttcctgagtttgtagcccccaggcgcagacgccccttacgccctgcctcccgcgtactgcgcggcgccagcgcgcggcgggaaacttgggcacagaaaacccctagagcaatttctgcttcccgaacttgttgcgctcctgtcccgcaacgcttccacgttccacgggctactgatattttaaaagtgagtgttcctcgggttgtgtgtggaaatattcattctcagtgcattttaggaaatcaagatgaaggccaaagggttaaggctaaaggcgttccgggcgtgcacctgtttggggaata

SEQ ID NO: 8 - DNA, 30 bp, Mus musculus
tgaagcttggtggcagattgagttcaagac

SEQ ID NO: 9 - DNA, 34 bp, Mus musculus
gcggatcccggtgcgggaacagcgccgggaccgg

SEQ ID NO: 10 - DNA, 30 bp, Mus musculus
gcggatccggattctgcgtcctgctcttcc

SEQ ID NO: 11 - DNA, 32 bp, Mus musculus
atgcggccgcatagccccagacacttggttcc

SEQ ID NO: 12 - DNA, 29 bp, Mus musculus
gatgggcgcatcgtaaccgtgcatctgcc

SEQ ID NO: 13 - DNA, 28 bp, Mus musculus
gagagatgggttccaggagctttggagc

SEQ ID NO: 14 - DNA, 30 bp, Mus musculus
gaagagcaggacgcagaatccggggcatcc

SEQ ID NO: 15 - DNA, 25 bp, Mus musculus
ggactacacgggcactgtacagttc

SEQ ID NO: 16 - DNA, 26 bp, Mus musculus
ggattcttgatcttcccaaactctgg

SEQ ID NO: 17 - DNA, 27 bp, Mus musculus
ccaagcttgcagagcgcctgtcacagg

SEQ ID NO: 18 - DNA, 29 bp, Mus musculus
ccttaattaactcctcacccctcacgctc

SEQ ID NO: 19 - DNA, 24 bp, Mus musculus
gcagctgcaggctcttgagtaggc

SEQ ID NO: 20 - DNA, 26 bp, Mus musculus
ctctgagaagatgctggatgaagagg

SEQ ID NO: 21 - DNA, 25 bp, Mus musculus
ggaagcctcacctgcgatgcgctcg

SEQ ID NO: 22 - DNA, 28 bp, Mus musculus
accatgaatctcaacttcacttcccctc

SEQ ID NO: 23 - DNA, 23 bp, Mus musculus
tcagagcacatgcggccctgagc

SEQ ID NO: 24 - DNA, 22 bp, Mus musculus
tgctaggtaacaagcgaatggg

SEQ ID NO: 25 - DNA, 20 bp, Mus musculus
agtccagcctagtggtggca

SEQ ID NO: 26 - DNA, 23 bp, Mus musculus
atgctgactgcaargttgctgag

SEQ ID NO: 27 - DNA, 21 bp, Mus musculus
tgcgactacagaggttcgtgg

SEQ ID NO: 28 - DNA, 19 bp, Mus musculus
tgctacctttgctgggtgc

SEQ ID NO: 29 - DNA, 19 bp, Mus musculus
tcacacagcttcccgatcc

SEQ ID NO: 30 - DNA, 18 bp, Mus musculus
gcaacatgtggaacgcga

SEQ ID NO: 31 - DNA, 20 bp, Mus musculus
aagcagatggcgaagtagcg

SEQ ID NO: 32 - DNA, 23 bp, Mus musculus
ttcagcttccaagtccataagga

SEQ ID NO: 33 - DNA, 23 bp, Mus musculus
aggtatcaaacttgccaacatgg

SEQ ID NO: 34 - DNA, 21 bp, Mus musculus
ttctacaaaggttccctgggc

SEQ ID NO: 35 - DNA, 20 bp, Mus musculus
accaagagactgacagcggc

SEQ ID NO: 36 - DNA, 20 bp, Mus musculus
ttgatcctgcactgaccagc

SEQ ID NO: 37 - DNA, 20 bp, Mus musculus
gattggcttgaggagttggc

SEQ ID NO: 38 - DNA, 19 bp, Mus musculus
tgcgactccaagatgcagg

SEQ ID NO: 39 - DNA, 21 bp, Mus musculus
ggcattaagccaccctcctct

SEQ ID NO: 40 - DNA, 19 bp, Mus musculus
ggctcccacaacatcacag

SEQ ID NO: 41 - DNA, 22 bp, Mus musculus
agctgaagcagaagtaacaggg

SEQ ID NO: 42 - DNA, 20 bp, Mus musculus
ctccgctctgcgacactaca

SEQ ID NO: 43 - DNA, 21 bp, Mus musculus
aatcagtgtctcagggctgga

SEQ ID NO: 44 - DNA, 20 bp, Mus musculus
gcggaggtgctagatccaca

SEQ ID NO: 45 - DNA, 21 bp, Mus musculus
aggactcgtgcagccttacac

SEQ ID NO: 46 - DNA, 20 bp, Mus musculus
acctcaccacggagagcaac

SEQ ID NO: 47 - DNA, 19 bp, Mus musculus
gcgagaggtcgagtttgca

SEQ ID NO: 48 - DNA, 19 bp, Mus musculus
cccgagccctggacatcta

SEQ ID NO: 49 - DNA, 21 bp, Mus musculus
gcttcgatctgcaacatagcg

SEQ ID NO: 50 - DNA, 20 bp, Mus musculus
ttcaccaccatggagaaggc

SEQ ID NO: 51 - DNA, 18 bp, Mus musculus
cccttttggctccaccct

SEQ ID NO: 52 - DNA, 18 bp, Mus musculus
caacgagcggttccgatg

SEQ ID NO: 53 - DNA, 19 bp, Mus musculus
cactgtgttggcatagagg

SEQ ID NO: 54 - DNA, 29 bp, Mus musculus
tctcatctcatcctgtgaaaccagtctgc

SEQ ID NO: 55 - DNA, 28 bp, Mus musculus
aagggaaatgggtcggaatccagcctgg

SEQ ID NO: 56 - DNA, 22 bp, Mus musculus
gcgcacaggtcgcagcaaggta

SEQ ID NO: 57 - DNA, 23 bp, Mus musculus
gcagaggtgctagatccacagaa

SEQ ID NO: 58 - DNA, 19 bp, Mus musculus
gcacaggagccaagagtga

SEQ ID NO: 59 - DNA, 19 bp, Mus musculus
agctccccaccatgttctg

SEQ ID NO: 60 - DNA, 17 bp, Mus musculus
cggaagggagcagccat

SEQ ID NO: 61 - DNA, 17 bp, Mus musculus
tcctggctctccctcct

SEQ ID NO: 62 - DNA, 15 bp, Mus musculus
gcagacagcatccag

SEQ ID NO: 63 - DNA, 16 bp, Mus musculus
cgatggaacatccagt

SEQ ID NO: 64 - DNA, 15 bp, Mus musculus
gcagccttcatatcg

SEQ ID NO: 65 - DNA, 14 bp, Mus musculus
gctctgtgatgttc

SEQ ID NO: 66 - DNA, 14 bp, Mus musculus
gcatggctcaccat

SEQ ID NO: 67 - DNA, 15 bp, Mus musculus
ccagccagcccagta

SEQ ID NO: 68 - DNA, 511 bp, Homo sapiens
ttgaccctattgaaggagctggatggatttagtcctccttatattaggaacactgtgatctgaataccacatccttaaatgttgttctcatgtgggttcacaccctaatttccactgcaaacatattatttttgaagacgcatttgtcagaagcagccagcaactccctttctttttgtagacatgtcatccactttggtagtgcttaaaataaagtactcagatagaaggtaagttgcaacagacaagcaatataatacaaaatgttatgagtaaagtgtgttactcagatacttctttcctttattttgtaggcaacttggctaatggaaccagttcttcctttgatcatcaatagagttatcttttcgggaaactaagtcccagctcagatgaccattttagctgaggctgacatgaaagggagagggactgtttaattattagctctctgaaaggcatgtttttttctttctttaataattcttcagctgaaacccaaacagagtac

SEQ ID NO: 69 - DNA, 521 bp, Mus musculus
  misc_feature (1)..(3): n is a, c, g, or t
nnnttgcccccagtgaaagaactgggtggatttagtctttgtcatgttagaaatgccgggttttcaatatcacatctttgaatgtgcgtctcatgagggttcacatcctcacttcccgtgcaaaagcatttgttttgaagatgcacttgctagaagcagccagcgactccagctctctttgcagacatgtcatccacttttgtagtgcttaaactaaagtactcagatagaaggtaggctgaaacagacaagcaatagaatacaaactgttacaagtaaagtgtgttactcaaatacgtgtctcctttattttgcgggcaacttggctaatggaaccagttcttcctttgatcatcaatagggccatctttttgggaaactaagtcccatctcagatggcagttttagctgaggctgacatgaaagagagaggggctgtttaattatccattccctgaaaagcacannnnnnnnctctctttaaattaatgattctccagctaaaacccaagcagaatgac

SEQ ID NO: 70 - DNA, 505 bp, Rattus norvegicus
cacccccaagtgagagaactgggtgggtttagtctttgtcacgttagaaacgccgggttttctatatcacatctttgaacgtgcttctcatgagggttcacatcctaacttcccgtgcaaaagcatttgttttgaagatgcatttgctagaagcagccagtgactctggttctttttgcagacatatcatccacttctgtagtgcttaaaataaagtactcagatagaaggtaggctggaacagacacacaggagaatacaaactgttaagagtagagtgcgttactcaaatacatgtctcctttattttgcgggcaacttggctaatggaactggttcttcctttgatcatcaacatggccatctttttgggaaactaagtcccatctcagatggcggttttagctgaggctgacatgaaagagagaggggctgtttaattatccattccctggaaagcacatcttttctctctttaaattagtgattctctagcagtaatgac

SEQ ID NO: 71 - DNA, 472 bp, Homo sapiens
cccacatcccttcctccttcctctctcttcatctacttaccagttcatcctcatggaatagaatgctcccacttcctggtgcactgtagtttggggcctaattgattctggtaattaatttgttctacctgctagatcagatggaaatcctacccatgcgccccaggattagcacttggctttctaacacctcctctaattatctaatagcatgggttgtgaggactcagtctctattaaaacacttttactattagcagtagtgattggaattgtgatgacgactagatttcaacaaattagagcttaaaaatggggagatgggagagaagtggcttcttctaatttccctgagtttaacatcaataattagagcaattttcagagatgtgagctgaaattacccctgctgtgttgtagatgatcaccctaattttagcaaattgactccagggaaataaatgtgacagat

SEQ ID NO: 72 - DNA, 475 bp, Mus musculus
tgccaggcttccttcctctgtttcctccccctatctgcttaccagtgtgtccgggtggtacaggacactcccacttcccggagcactgaagtttggggcctaattgattctggtaattaatttgttctacctgctagatcagatggaaatcctactgcagcgcccgaggattaacacttgacttcgcagcacctctgctaattatctaatagcatgggttgtgaggactcagtctctgttaaaacacttttgctattagctgtagtgattggaattgtgatgacgactagatatcaacaaattagagcttaaaaatgaggagatggaagagaagtggcttcttttaatttccccgaatttaacatcaataattagagcaattttcagagatgcgagcccaaattactcctgctgtgctgtagatgatcaccttaattttagcaaattgactccagggaaataaatgtgacagatt

SEQ ID NO: 73 - DNA, 478 bp, Rattus norvegicus
  misc_feature (1)..(1): r is g or a
rattsnrwgcsagccatgcttccttccactgtttcctccccttcccagagcgtccgtgtggagcaggatgcttccacttcctggagcactgaagtttggggcctaattgattctggtaattaatttgttctacctgctagatcagatggaaatcctactgcagcgcccgaggattaacacttgacttcgcagcaccctcgctaattatctaatagcatgggttgtgaggactcagtctctgttaaaacacttttgctattagcagtagtgattggaattgtgatgacgactagatatcaacaaattagagcttaaaaatggggagatggaagagaagtggcttcttttaatttccccgagtttaacatcaataattagagcaattttcagagatgcgagcccaaattactcctgctgtgctgtagatgatcaccttaattttagcaaattaactccagggaaataaatgtgacagatt

SEQ ID NO: 74 - DNA, 177 bp, Homo sapiens
ggctcgcagggcaaaactgggaatgatcacgcgtttccaggaatggtttgccaccgcgttctcctacgcgtacgccccctcccctgccctaggcagtttcctagtccctgtagaaatgaacttgcgaataacctttcattaactgatcccctccctctcactccaaattttctgttt

SEQ ID NO: 75 - DNA, 191 bp, Mus musculus
tagataataaaggacttgtggggcaaaaaatggaaaggagcgcaggtttccgggaaccgtttgttgccacgtcccgctgctagcacatcacctcccttgtcccagacactttgttattcccaaaagaaatgaatttggctaataacctttacttaacagattccgcctccctgaccccgactttcctgttt

SEQ ID NO: 76 - DNA, 189 bp, Rattus norvegicus
tagataataaaggacttgtggggcaaaaatgggaggagcacgggtttccgggaatcatttgttgtcgcgtcccgttgcttgcacgtcacctccctcatcccaggcagtttcttattcccagtagaaatgaatttggctagtgacctttacttaacagattccgtctccctgaccccgagtttcctgttt